(19) United States
(12) Patent Application Publication
(10) Pub. No.: US 2024/0252219 A1
(43) Pub. Date: Aug. 1, 2024

(54) LAPIDUS TOTAL REDUCTION CLAMP

(71) Applicant: Acumed LLC, Hillsboro, OR (US)

(72) Inventors: Joseph Pepin, Sachse, TX (US); Roland Shamenderaj Ramdass, Winchester, VA (US)

(21) Appl. No.: 18/428,108

(22) Filed: Jan. 31, 2024

Related U.S. Application Data
(60) Provisional application No. 63/442,663, filed on Feb. 1, 2023.
(60) Provisional application No. 63/524,410, filed on Jun. 30, 2023.

(51) Int. Cl. A61B 17/88; A61B 17/17; A61F 2/46

(52) U.S. Cl. CPC A61B 17/8866; A61B 17/1775; A61F 2/4606

(57) ABSTRACT

A system includes a main correction assembly having a fixation body configured to be fixed to a first bone of a patient, a movable body configured to move relative to the fixation body. The movable body includes an anchor body configured to be fixed to a second bone of the patient, a first portion configured to rotate the anchor body relative to the fixation body in a first rotating direction in a first plane. The main correction assembly defines a rotation axis about which the anchor body is rotated, by the first portion, relative to the fixation body in the first plane. The main correction assembly is configured such that the rotation axis is disposed substantially within a joint adjacent the second bone when the fixation body is fixed to the first bone.

### PRIORITY CLAIM

This application claims priority to U.S. Provisional Patent Application No. 63/442,663 filed on Feb. 1, 2023 and U.S. Provisional Patent Application No. 63/524,410 filed on Jun. 30, 2023, the entire contents of each of which are hereby incorporated by reference and relied upon.

### BACKGROUND

Lapidus procedure refers to a surgical procedure for the treatment of a bunion deformity. A bunion is a bony bump that is formed on the side of the big toe joint, which may cause a foot deformity. Lapidus procedure involves the correction of the foot structure by repositioning the poorly aligned first metatarsal bone to its proper position.

Lapidus procedures traditionally require full exposure of the first tarsometatarsal (TMT) joint. This means that traditionally an incision having a length of at least 3.5 cm is needed to complete the Lapidus procedure, which may leave a big scar, require more healing time, and have a risk of infection.

### SUMMARY

The present disclosure provides new and innovative systems and methods for controlled realignment of a bone (e.g., first metatarsal bone) in multiple planes for a Lapidus surgery. In some examples, a system includes a main correction assembly having a fixation body configured to be fixed to a first bone of a patient and a movable body configured to move relative to the fixation body. The movable body includes an anchor body configured to be fixed to a second bone of the patient and a first portion having a first moving mechanism configured to move the anchor body relative to the fixation body in a first direction in a first plane.

In some examples, a method according to the present disclosure may include aligning the main correction assembly with respect to the first and second bones of the patient, fixing the fixation body to the first bone of the patient, fixing the anchor body to the second bone of the patient, moving the anchor body, using the first moving mechanism, relative to the fixation body in the first direction in the first plane to correct a position of the second bone in the first direction, and cutting the second bone by a bone cutting device.

Additional features and advantages of the disclosed systems and methods are described in, and will be apparent from, the following Detailed Description and the Figures.

### BRIEF DESCRIPTION OF THE FIGURES

FIG. 1 is a diagram of a perspective view of a system for controlled realignment of a bone in multiple planes according to an example of the present disclosure.

FIG. 2 is a diagram of a perspective view of an example main correction assembly of the system of FIG. 1.

FIG. 3 is a diagram of a right side view of the main correction assembly of FIG. 2.

FIG. 4 is a diagram of a left side view of the main correction assembly of FIG. 2.

FIG. 5 is a diagram of a rear side view of the main correction assembly of FIG. 2.

FIG. 6 is a diagram of a portion of an example fixation body of the main correction assembly of FIG. 2.

FIG. 7 is a diagram of a perspective view of an example alignment guide of the system of FIG. 1.

FIG. 8 is a diagram of a perspective view of an example cutting guide of the system of FIG. 1.

FIG. 9 is a diagram of a perspective view of another example cutting guide of the system of FIG. 1.

FIGS. 10A and 10B are diagrams of a system for controlled realignment of a bone in multiple planes according to another example of the present disclosure.

FIG. 11 is a diagram illustrating a method for controlled realignment of a bone in multiple planes.

FIG. 12 is a diagram illustrating a method for controlled realignment of a bone in multiple planes.

FIG. 13 is a diagram illustrating a method for controlled realignment of a bone in multiple planes.

FIG. 14 is a diagram illustrating a method for controlled realignment of a bone in multiple planes.

FIG. 15 is a diagram illustrating a method for controlled realignment of a bone in multiple planes.

FIG. 16 is a diagram illustrating a method for controlled realignment of a bone in multiple planes.

FIG. 17 is a diagram of a top view of an anatomical structure of a foot of a patient with bunion.

FIG. 18 is a diagram of a front-right perspective view of a system for controlled realignment of a bone in multiple planes according to another example of the present disclosure.

FIG. 19 is a diagram of a top view of the system of FIG. 18.

FIG. 20 is a diagram of a front-rear perspective view of the system of FIG. 18.

FIG. 21 is a diagram of a fixation body of the system of FIG. 18.

FIGS. 22A-C are diagrams of various cross-sectional views of the fixation body of the system of FIG. 18.

FIG. 23 is a diagram of an expanded view of the fixation body and a first portion of the system of FIG. 18.

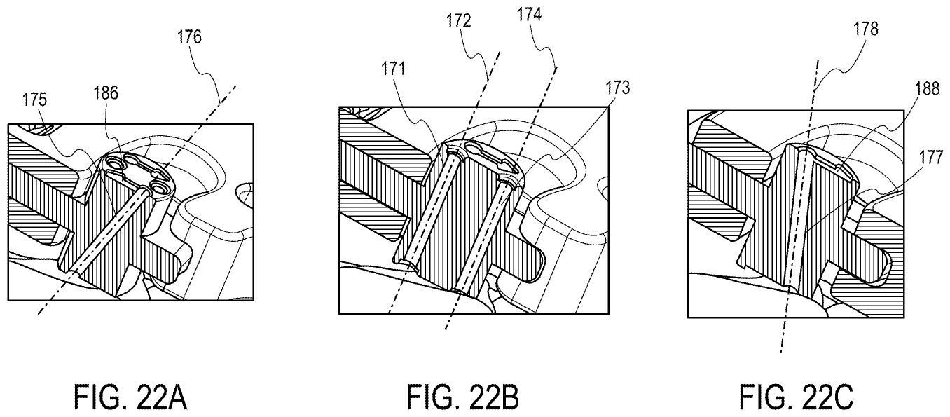

FIG. 24 is a diagram of a front-right perspective view of a system for controlled realignment of a bone in multiple planes according to another example of the present disclosure.

FIG. 25 is a diagram of a front-right perspective view of an example main correction assembly of the system of FIG. 24.

FIG. 26 is a diagram of a front-right perspective view of an example cutting guide assembly of the system of FIG. 24.

FIG. 27 is a diagram of a perspective view of an example cutting guide according to an example of the present disclosure.

FIG. 28 is a diagram of a perspective view of another example cutting guide according to an example of the present disclosure.

FIGS. 29A and 29B illustrate an example usage of the cutting guides of FIGS. 27 and 28, respectively, according to an example of the present disclosure.

FIGS. 30A and 30B are a diagram of a perspective view of another example cutting guide according to an example of the present disclosure.

FIGS. 31A and 31B illustrate an example usage of the cutting guide of FIGS. 30A and 30B according to an example of the present disclosure.

FIG. 32 is a diagram of a front-right perspective view of a system for controlled realignment of a bone in multiple planes according to another example of the present disclosure.

FIG. 33 is a diagram of a side view of the system of FIG. 32.

### DETAILED DESCRIPTION OF EXAMPLE EMBODIMENTS

The present disclosure is directed to systems and methods for controlled realignment of a bone (e.g., first metatarsal bone) in multiple planes, for example, for a Lapidus surgery. As discussed above, the conventional Lapidus procedure may require an incision having a length of at least 3.5 cm, which may have various issues such as a big scar, more healing time, and infection risk. Moreover, conventional devices for the Lapidus procedure may only allow a single (sagittal) plane correction, requiring the surgeon to perform the correction in other planes (e.g., frontal and transverse planes) manually, which may necessitate a bigger incision size, and reduce the accuracy and repeatability of the procedure.

Aspects of the present disclosure may address the above-discussed issues in the conventional Lapidus procedure. For example, aspects of the present disclosure may provide a (single) system/device having multiple moving mechanisms, which allows a multi-planar (sagittal, frontal, and transverse planes) correction of the bone (e.g., first metatarsal bone) as well as compressing/distracting of the joint (e.g., first tarsometatarsal joint) adjacent the bone to be corrected. Moreover, since the bone can be corrected in multiple planes using the system according to the present disclosure, the accuracy and repeatability of the Lapidus procedure can be improved.

Moreover, the system according to the present disclosure may allow the surgeon to make two (parallel) cuts near the joint (e.g., first tarsometatarsal joint) between two bones to be cut, which may enable the Lapidus surgery to be performed with a smaller incision (e.g., around 3 to 3.5 mm) than the traditional approaches (e.g., having an approximately 3.5 cm incision). This may reduce the size of the scar, healing times, and risk of infection compared to the traditional full exposure techniques.

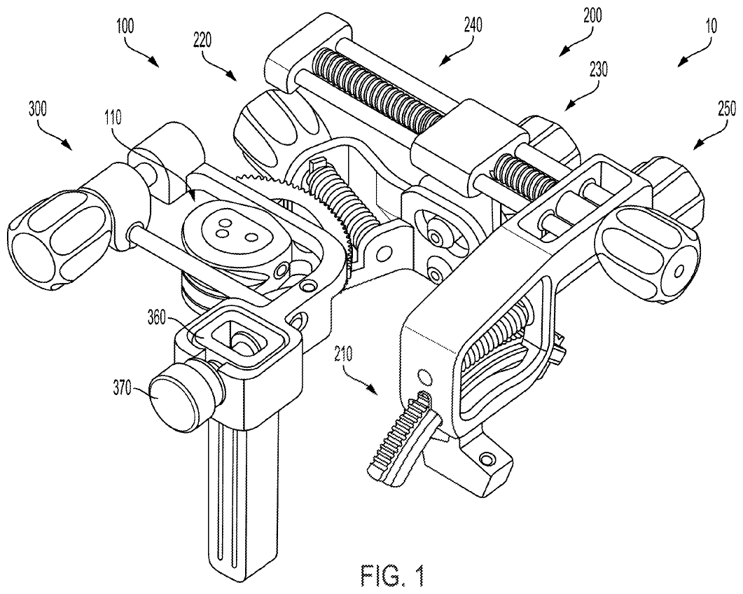

FIG. 1 depicts an example system 10 for controlled realignment of a bone in multiple planes according to an example of the present disclosure. As shown in FIG. 1, the system 10 may include a main correction assembly 100 and an alignment guide 300.

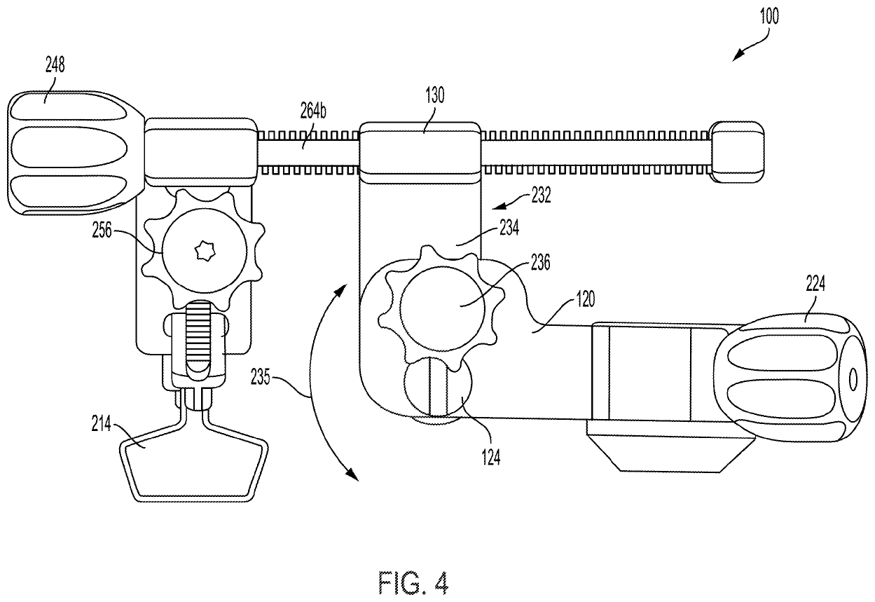

Referring to FIGS. 1 to 5, the main correction assembly 100 may include a fixation body 110 and a movable body 200.

The fixation body 110 may be configured to be fixed to a first bone of a patient. In some examples, the first bone may be an intermediate cuneiform bone. In other examples, the first bone may be a lateral cuneiform bone, a cuboid bone, a navicular bone, or any other suitable and/or stable bone of a patient.

The movable body 200 may be configured to move relative to the fixation body 110. The movable body 200 may include an anchor body 210. The anchor body 210 may be configured to be fixed to a second bone of the patient. In some examples, the second bone may be a first metatarsal bone. In other examples, the second bone may be any other bone (e.g., other metatarsal bones) that needs to be corrected.

The movable body 200 may further include a first portion 220 having a first moving mechanism 222. The first moving mechanism 222 may be configured to move the anchor body 210 relative to the fixation body 110 in a first (rotational) direction 225. In some examples, the first direction 225 may be in a first plane. The first plane may be a transverse plane or substantially parallel to the transverse plane (for example, when the main correction assembly 100 is attached to a human body).

In some examples, the fixation body 110 may include an engagement feature 114 configured to be engaged with the first moving mechanism 222. For example, the first moving mechanism 222 may be rotationally coupled to the engagement feature 114 of the fixation body 110. In some examples, the engagement feature 114 and the first moving mechanism 222 together may form a gear arrangement. For example, the first moving mechanism 222 may be in the form of a worm/threaded rod/screw, and the engagement feature 114 may be in the form of a worm wheel (at least a partial worm wheel) or toothed flange. In other examples, the engagement feature 114 and the first moving mechanism 222 together may form any other suitable arrangement/adjustment mechanism, through which the anchor body 210 may move/rotate relative to the fixation body 110 in the first direction 225 in the first plane.

The first portion 220 may further include a graspable device 224 (e.g., knob) connected to the first moving mechanism 222. A user (e.g., a surgeon) can use the graspable device 224 to rotate the first moving mechanism 222, for example, by rotating the graspable device 224. The rotation of the first moving mechanism 222 may rotate anchor body 210 relative to the fixation body 110 in the first direction 225 in the first plane. In some examples, the rotation of the first moving mechanism 222 may further rotate all components disposed between the first moving mechanism 222 and the anchor body 210 in the first direction 225 in the first plane.

In some examples, the movable body 200 may include a second portion 230. The second portion 230 may have a second moving mechanism 232 configured to move and/or rotate the anchor body 210 relative to the fixation body 110 in a second direction 235 in a second plane. The second moving mechanism 232 may include a second portion body 234. In some examples, the second moving mechanism 232 may further move all components disposed between the second moving mechanism 232 and the anchor body 210 in the second direction 235 in the second plane. In some examples, the second plane may be perpendicular to the first plane. The second plane may be a sagittal (dorsal/plantar) plane or substantially parallel to the sagittal plane (for example, when the main correction assembly 100 is attached to a human body).

In some examples, the second portion 230 may be coupled to the first portion 220 via a first connecting body 120. The first connecting body 120 may include a first plate 120a and a second plate 120b. Each of the first plate 120a and the second plate 120b may include a hole configured to receive a rotating rod 124.

In some examples, at least a portion of the second portion body 234 may be disposed between the first plate 120a and the second plate 120b. In some examples, at least a portion of the second portion body 234 may extend out from the first and/second plates 120a, 120b. The second portion body 234 may include a hole configured to receive the rotating rod 124. The rotating rod 124 may define a longitudinal axis 125, and the second portion body 234 may rotate around the longitudinal axis 125 of the rotating rod 124, which serves as a pivot axis. In some examples, the second portion body 234 of the second portion 230 may be in a tower (e.g., rectangular) shape. In other examples, the second portion body 234 may have any other suitable shape (e.g., as long as it can be rotated around the pivot axis 125).

In some examples, the second portion may include a locking mechanism 237. The second portion body 234 may include a hole configured to receive the locking mechanism 237. In some examples, the locking mechanism 237 may be in a rod shape. In other examples, the locking mechanism 237 may have any other suitable shape.

The first and second plates 120a, 120b may include a recess 122, in which the locking mechanism 237 may be placed. The locking mechanism 237 may be able to move within the recess 122. The recess 122 together with the locking mechanism 237 may define a range of rotation 231 of the second portion 230 (e.g., the second portion body 234).

In some examples, the locking mechanism 237 and the second portion body 234 may have a threaded engagement. The locking mechanism 237 may include a compressing portion 239 that may be disposed outside of the first and second plates 120a, 120b and facing the second plate 120b. The compressing portion 239 may have a diameter greater than the diameter of other portions of the locking mechanism 237. When the locking mechanism 237 is rotated in one direction (e.g., counterclockwise), the compressing portion 239 of the locking mechanism 237 may move toward the second plate 120b and compress the second plate 120b against the second portion body 234, thereby locking the movement of the second portion 230 (e.g., the second portion body 234). When the locking mechanism 237 is rotated in another direction (e.g., clockwise), the compressing portion 239 of the locking mechanism 237 may move away from the second plate 120b and release the second plate 120b from the second portion body 234, thereby unlocking the second portion 230 so that the second portion body 234 can be rotated around the pivot axis 125. The locking mechanism 237 of the second portion 230 can prevent the anchor body 210 from being rotated in the second plane unintentionally, for example, after the second bone is corrected in the second (sagittal) plane.

In some examples, the second portion 230 may further include a graspable device 236 (e.g., knob) connected to the locking mechanism 237. A user (e.g., a surgeon) can use the graspable device 236 to rotate the first locking mechanism 237, for example, by rotating the graspable device 236.

In some examples, the movable body 200 may include a third portion 240. The third portion 240 may have a third moving mechanism 242. The third moving mechanism 242 may be configured to move the anchor body 210 relative to the fixation body 110 in a third direction 245. In some examples, the third moving mechanism 242 may further move all components disposed between the third moving mechanism 242 and the anchor body 210 in the third direction 245. In some examples, the third direction 242 may be disposed in the first plane and/or the second plane.

In some examples, the third portion 240 may be coupled to the second portion 230 via a second connecting body 130. For example, the second connecting body 130 may be connected to one end portion of the second portion body 234 (opposite the portion of the second portion body 234 disposed between the first plate 120a and the second plate 120b). The second connecting body 130 may include a hole 132 configured to receive the third moving mechanism 242. The second connecting body 130 and the third moving mechanism 242 may have a threaded engagement. For example, the third moving mechanism 242 may include a thread, and the hole 132 of the second connecting body 130 may include an internal thread. The rotation of the third moving mechanism 242 may move the third moving mechanism 242 (and ultimately the third portion 240) relative to the second connecting body 130 in the third direction 245.

In some examples, the third moving mechanism 240 may be in a shape of a rod (e.g., threaded rod). In other examples, the third moving mechanism 240 may have any other suitable shape. In some examples, the third portion 240 may further include one or more support bodies 246a and 246b that may extend in parallel with the third moving mechanism 240. In this case, the second connecting body 130 may further include one or more holes 134a, 134b configured to receive the one or more support bodies 246a and 246b.

The rotation of the third moving mechanism 240 may move the anchor body 210 (and all the other components between the third moving mechanism 240 and the anchor body 210) in the third direction 245. The movement of the anchor body 210 relative to the fixation body 110 in the third direction 245 may cause a joint adjacent the second bone (e.g., first tarsometatarsal joint) to be compressed or distracted. In other examples, the third moving mechanism 240 may move back and forth (in the third direction 245) in any other suitable manner (e.g., simply pushing or pulling the moving mechanism 240 back and forth).

In some examples, the third portion 240 may further include a graspable device 248 (e.g., knob) connected to the third moving mechanism 242. A user can use the graspable device 248 to rotate the third moving mechanism 242, for example, by rotating the graspable device 248.

In some examples, the third portion 240 may include a base cap 247. The base cap 247 may cover one end portion of each of the third moving mechanism 242 and/or the one or more support bodies 246a, 246b (opposite the end portion adjacent the graspable device 248).

In some examples, the movable body 200 may further include a fourth portion 250. The fourth portion 250 may include a fourth moving mechanism 252. The fourth moving mechanism 252 may be configured to move and/or rotate the anchor body 210 relative to the fixation body 110 in a fourth direction 255 in a third plane. In some examples, the fourth moving mechanism 252 may further move and/or rotate all components disposed between the fourth moving mechanism 252 and the anchor body 210, if any, in the fourth direction 255 in the third plane. In some examples, the third plane may be perpendicular to the first plane and/or the second plane. The third plane may be a frontal plane or substantially parallel to the frontal plane (for example, when the main correction assembly 100 is attached to a human body).

In some examples, the fourth portion 250 may be coupled to the third portion 240 via a third connecting body 140. The third connecting body 140 may include a first portion 141 and a second portion 143. One end portion of each of the third moving mechanism 242 and/or the one or more support bodies 246a, 246b (opposite the portion covered by the base cap 247) may be connected to the first potion 141 of the third connecting body 140. For example, the first portion 141 may include one or more holes 142a, 142b, 142c configured to receive the third moving mechanism 242 and/or the one or more support bodies 246a, 246b.

The second portion 143 of the third connecting body 140 may include one or more holes 144a, 144b configured to receive the fourth moving mechanism 252. The second portion 143 may include an internal space 147 in which at least a portion of the fourth moving mechanism 252 may be disposed.

In some examples, the fourth moving mechanism 252 may be in a rod shape (at least a portion of the rod being threaded). In other examples, the fourth moving mechanism 252 may have any other suitable shape.

In some examples, the fourth portion 250 may further include a graspable device 256 (e.g., knob) connected to the fourth moving mechanism 252. A user can use the graspable device 256 to rotate the fourth moving mechanism 252, for example, by rotating the graspable device 256.

In some examples, the anchor body 210 may include a (toothed) rotating arm 212. The rotating arm 212 may be rotationally coupled to the fourth moving mechanism 252. In some examples, the rotating arm 212 and the fourth moving mechanism 252 together may form a gear arrangement. For example, the fourth moving mechanism 252 may in the form of a worm/threaded rod/screw, and the rotating arm 212 may be in the form of a worm wheel (at least a partial worm wheel)/toothed flange. In other examples, the rotating arm 212 and the fourth moving mechanism 252 together may form any other suitable arrangement/adjustment mechanism, through which the anchor body 210 may rotate relative to the fixation body 110 in the fourth direction 255 in the third plane.

In some examples, the second portion 143 of the third connecting body 140 may include one or more recesses 145a, 145b configured to receive the rotating arm 212. In some examples, at least a portion of the rotating arm 212 may be disclosed in the internal space 147 of the second portion 143.

In some examples, the rotating arm 212 may include one or more side protrusions 213a, 213b. Each of the one or more recesses 145a, 145b may include a corresponding recess 146a, 146b configured to receive the one or more side protrusions 213a, 213b of the rotating arm 212.

In some examples, the anchor body 210 may include an anchor base 214. The anchor base 214 may be configured to be fixed to the second bone of the patient. In some examples, the anchor base 214 may be fixed to a distal portion of the second bone. In other examples, the anchor base 214 may be fixed to any other suitable portion (middle or proximal portion) of the second bone.

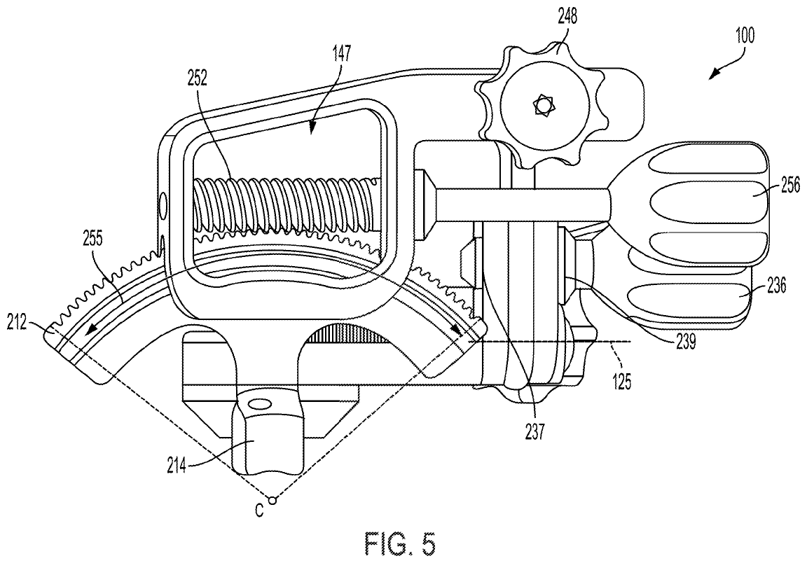

In some examples, the rotating arm 212 (at least the portion adjacent and/or to be engaged with the fourth moving mechanism 252) may be in a shape of a curve or a partial circle. In some examples, as shown in FIG. 5, the rotating arm 212 may be configured so that a center C of the curve or the partial circle may be located in the second bone when the anchor body 210 is fixed to/placed on the second bone. The rotation of the fourth moving mechanism 250 may rotate the rotating arm 212 (and ultimately the second bone) around the center C, which may serve as a rotational axis.

In some examples, the shortest distance between the anchor base 214 and the center C of the curve of the rotating arm 212 may be in a range of about 0.3 cm to about 1.5 cm, for example, about 0.3 cm to about 0.7 cm, about 0.7 cm to about 1.2 cm, or about 1.2 cm to about 1.5 cm. In other examples, the shortest distance between the anchor base 214 and the center C of the curve may have any other suitable value (e.g., less than 0.3 cm or greater than 1.5 cm).

In some examples, the anchor base 214 may extend in a direction (substantially) parallel to the third direction 245. In some examples, the rotating arm 212 may extend in a direction (substantially) perpendicular to the third direction 245. When the anchor body 210 is fixed to the second bone, the longitudinal direction of the second bone may be (substantially) parallel to the third direction 245.

In some examples, the anchor base 214 may include one or more channels 216a, 216b configured to receive a bone fastener. When there are multiple channels, the channels may be distributed in the third direction 245 along the anchor base 214. In some examples, the bone fastener may be a pin, a screw, a tack, or a k-wire. In other examples, the bone fastener may be any other suitable fastening device that can (temporally or removably) fix the target component (e.g., anchor base, fixation body) to the bone of a patient. In some examples, the bone fastener may include a hat that may prevent the bone fastener from penetrating into the bone deeper than a predetermined length (so that it does not penetrate into another bone or tissue unintentionally). In some examples, the diameter of the bone fastener may be smaller than the diameter of the one or more channels 216a, 216b.

In other examples, the anchor base 214 may have any other suitable fixing mechanism, such as a clamp or any other suitable device that can (temporally or removably) fix the anchor base 214 to the second bone of a patient.

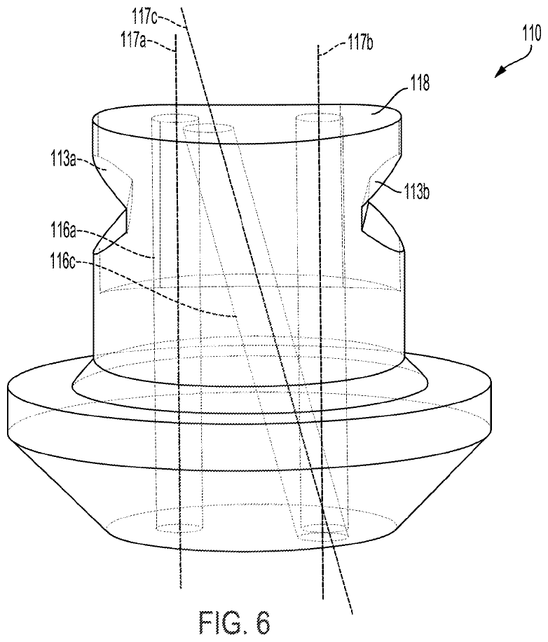

In some examples, the fixation body 110 may include an internal body 118 and an external body 119. The external body 119 may include the engagement feature 114. FIG. 6 illustrates a portion (including the internal body 118) of an example fixation body 110 of a main correction assembly according to the present disclosure. In some examples, the fixation body 110 may include one or more channels configured to receive a bone fastener. Referring to FIG. 6, in some examples, the one or more channels of the fixation body 110 may include a first channel 116a having a first trajectory 117a and a second channel 116b having a second trajectory 117b. In some examples, the first and second trajectories 117a, 117b may be parallel to each other. In this way, after the fixation body 110 is fixed to the first bone, the main correction assembly 100 can still be moved along the first/second trajectory 117a/117b (e.g., in a dorsal to plantar or plantar to dorsal direction).

In some examples, the one or more channels of the fixation body 110 may include a third channel 116c having a third trajectory 117c. The third trajectory 117c may be non-parallel to the first and second trajectories 117a, 117b. Therefore, when bone fasteners are inserted into the third channel 116c and at least one of the first and second channels 116a, 116b, the movement of the main correction assembly 100 along the first/second trajectory 117a/117b (e.g., in a dorsal to plantar or plantar to dorsal direction) may be prevented. The angle between the first/second trajectory 117a/117b and the third trajectory 117c may be in a range of about 5° to about 25°. In other examples, the angle between the first/second trajectory 117a/117b and the third trajectory 117c may have any other suitable value (less than 5° or greater than 25°). In some examples, the diameter of the bone fastener may be smaller than the diameter of the one or more channels 116a, 116b, 116c.

In some examples, as shown in FIG. 6, the internal body 118 of the fixation body 110 may include one or more grooves 113a, 113b configured to receive a fastener (e.g., a screw) to fix the internal body 118 to the external body 119. The fastener to fix the internal body 118 to the external body 119 may be inserted via a through-hole 111 formed on the external body 119 and received in the one or more grooves 113a, 113b of the internal body 118. In some examples, the fastener may be in a threaded engagement with the through-hole 111 and/or the one or more grooves 113a, 113b.

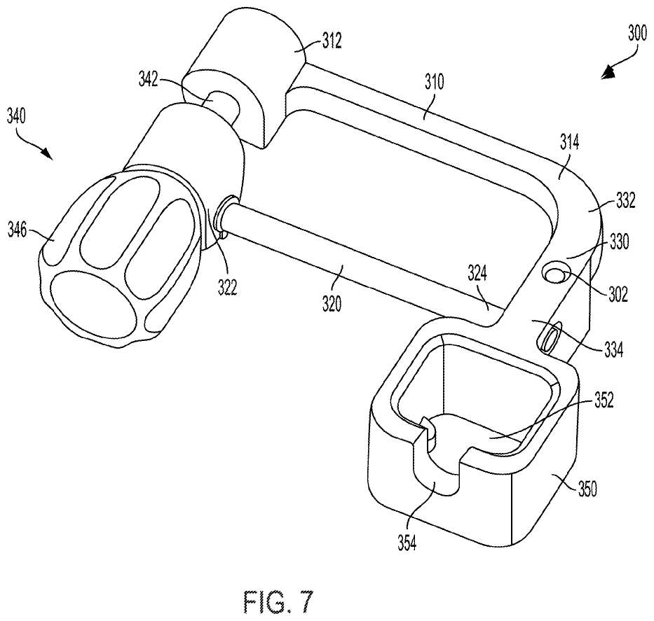

In some examples, the alignment guide 300 may be removably coupled to the fixation body 110. In other examples, the alignment guide 300 may be permanently coupled to the fixation body 110. Referring to FIGS. 1 and 7, in some examples, the alignment guide 300 may include an alignment channel 302. The alignment channel 302 may be provided to receive a bone fastener. The alignment guide 300 and the main correction assembly 100 may be aligned with respect to the first bone and the second bone using the alignment channel 302 and the bone fastener. For example, the bone fastener may be inserted into a joint (e.g., first tarsometataral joint) adjacent the second bone of the patient, and the bone fastener may be inserted down through the alignment channel 302. In other examples, the alignment channel may be provided on the main correction assembly 100. In some examples, the diameter of the bone fastener may be smaller than the diameter of the alignment channel 302.

In some examples, the alignment guide 300 may further include a first support 310, a second support 320, and/or a third support 330. The first support 310 may have a first end portion 312 and a second end portion 314. The second support 320 may have a first end portion 322 and a second end portion 324. The fixation body 110 may be interposed between the first support 312 and the second support 312 when the alignment guide 300 is coupled to the fixation body 110. The third support 330 may have a first end portion 332 connected to the second end portion 314 of the first support 310 and a second end portion 334 connected to the second end portion 324 of the second support 320.

In some examples, the alignment guide 300 may further include a securing device 340 configured to secure the alignment guide 300 to the fixation body 110. The securing device 340 may include a shaft 342 and a graspable device 346 (e.g., knob). The securing device 340 (e.g., the shaft 342) may be connected to the first end portion 312 of the first support 310 and the first end portion 322 of the second support 320. When the shaft 342 of the securing device 340 is rotated in one direction (e.g., counterclockwise), for example, using the graspable device 346, the first end portion 322 of the second support 320 may move toward the first end portion 312 of the first support 310, thereby securing the fixation body 110 with the assignment guide 300 by compressing the fixation body between the first support 310 and the second support 320. When the shaft 342 of the securing device 340 is rotated in another direction (e.g., clockwise), the first end portion 322 of the second support 320 may move away from the first end portion 312 of the first support 310, thereby loosening the engagement between the fixation body 110 and the assignment guide 300.

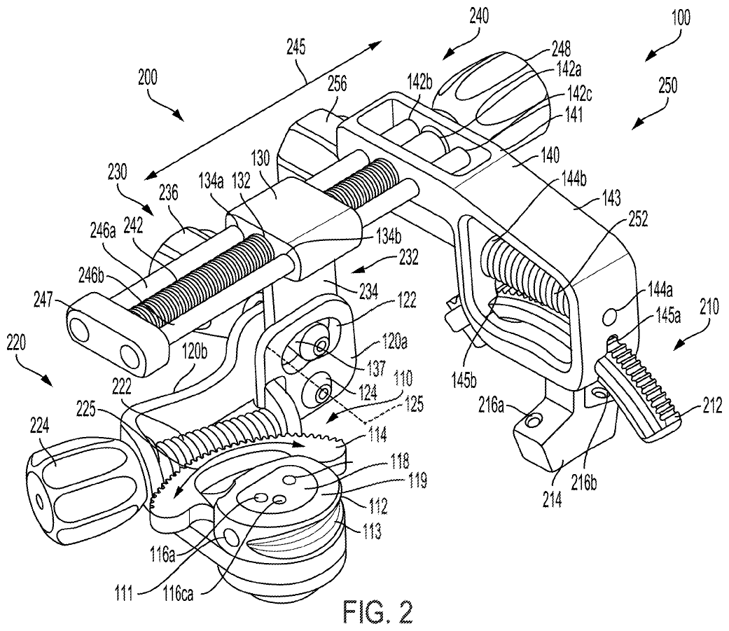

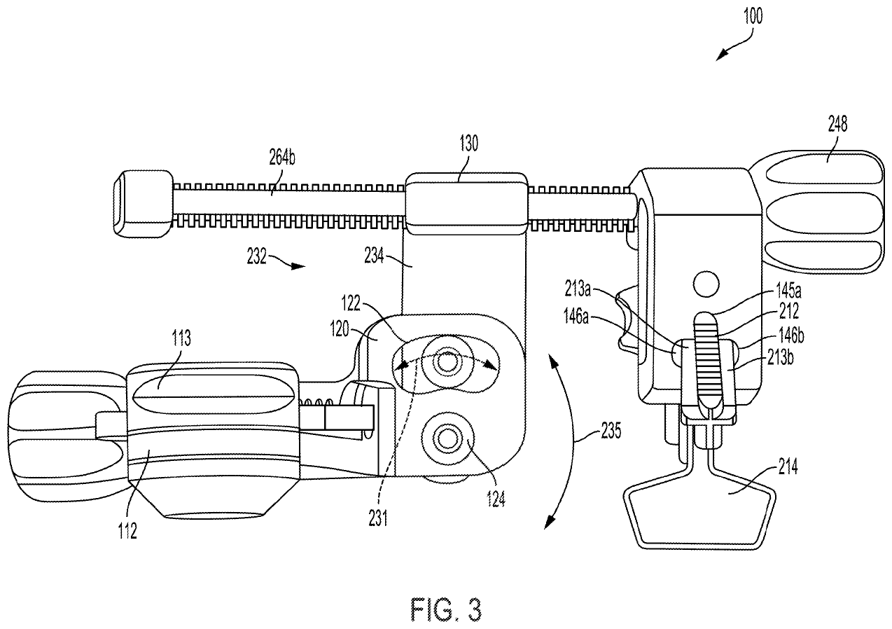

In some examples, the alignment guide 300 may be coupled to the fixation body 110 in a way that allows the alignment guide 300 to rotate and/or translate relative to the fixation body 110, for example, in the first (transverse) plane. For example, referring back to FIGS. 2 and 3, in some examples, the fixation body 110 may include one or more grooves 113. The one or more grooves 113 may be configured to receive the second support 320. The one or more grooves 113 may be provided so that the alignment guide 300 can be easily attached to, removed from, and/or rotated around the fixation body 110. The one or more grooves 113 and the second support 320 are sized and shaped so that the second support 320 can be rotated over the one or more grooves 113.

In some examples, the alignment channel 302 may be disposed in the third support 330. In other examples, the alignment channel 302 may be disposed in any other suitable component of the alignment guide 300 (e.g., first support 310, second support 320).

In some examples, the alignment guide 300 may further include a receiver 350. The receiver 350 may be provided to receive a cutting guide 360. The cutting guide 360 may be provided to guide a bone cutting device. The receiver 350 may be disposed at the second end portion 334 of the third support 330. The receiver 350 may include a cutting guide receiving hole 352 and a fastener receiving hole 354.

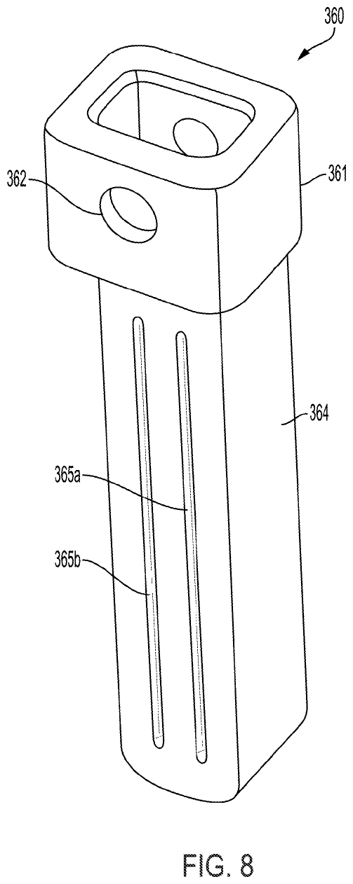

FIG. 8 illustrates an example cutting guide 360 according to an example of the present disclosure. In some examples, the cutting guide 360 may include a head portion 361 and a body portion 364. The head portion 361 may be sized and shaped to be received in the cutting guide receiving hole 352 of the receiver 350. The head portion 361 may include a fastener receiving hole 362. In some examples, a fastener 370 may be provided to fix the cutting guide 360 to the receiver 350. For example, the fastener 370 can be inserted into the fastener receiving hole 354 of the receiver 350 and the fastener receiving hole 362 of the cutting guide 360.

The cutting guide 360 may include one or more cutting slots 365a, 365b. A bone cutting device may be inserted into the one or more cutting slots 365a, 365b to cut the second bone and/or a third bone. For example, the bone cutting device may be inserted into a first cutting slot 365a to cut the second bone and into the second cutting slot 365b to cut the third bone. The third bone may be a medial cuneiform bone.

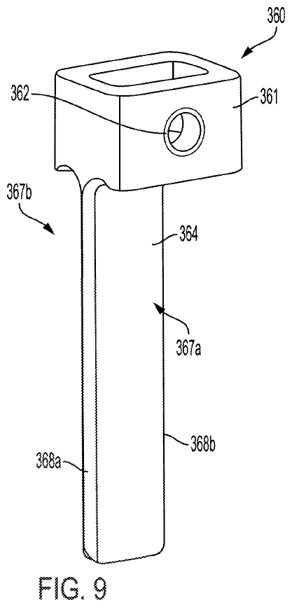

FIG. 9 illustrates another example of the cutting guide 360 according to an example of the present disclosure. The cutting guide 360 may include one or more guiding surfaces 367a, 367b. A bone cutting device may be abutted adjacent to the one or more guiding surfaces 367a, 367b to cut the second bone and/or the third bone. For example, the bone cutting device may be abutted adjacent a first guiding surface 367a to cut the second bone, and abutted adjacent a second guiding surface 367b to cut the third bone. Examples of the bone cutting device may include a bur, a surgical saw (e.g., sagittal saw), an osteotome, and a reciprocating saw.

In some examples, the cutting guide 360 may serve as a joint alignment guide.

In this case, the cutting guide 360 may include one or more ridges configured to be aligned with a joint (e.g., first tarsometatarsal joint) adjacent the second bone of the patient. For example, the cutting guide 360 may include one or more side edges 368a, 368b, which may serve as the ridge. Other configurations/features/characteristics of the cutting guide 360 of FIG. 9 may be similar to and/or same as the ones described above with respect to the cutting guide 360 illustrated with respect to FIG. 8 and, thus, duplicate description may be omitted.

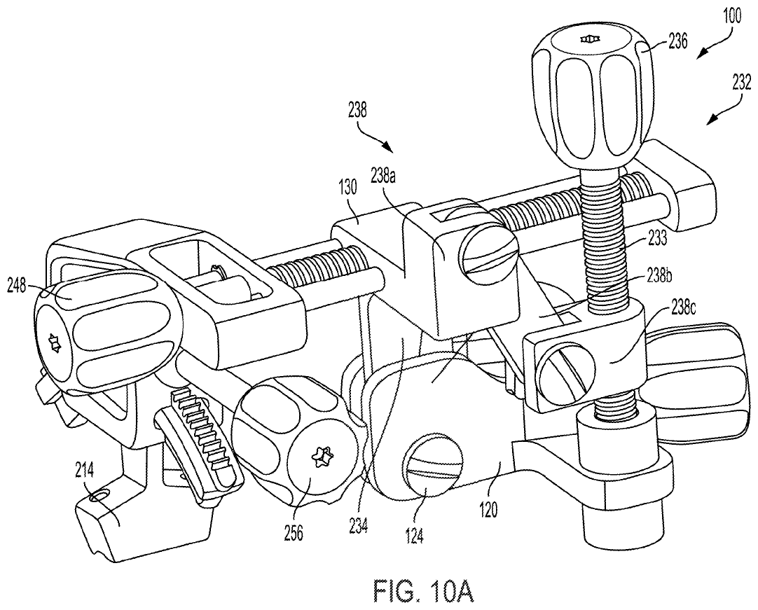

FIGS. 10A and 10B illustrate another example of a main correction assembly 100 according to an example of the present disclosure. The second portion 230 may have a second moving mechanism 232. The second moving mechanism 232 may be configured to move and/or rotate the anchor body 210 relative to the fixation body 110 in the second direction 235 in the second plane. In this example, the second moving mechanism 232 may include a second portion body 234, a mover 233, and a connector 238 disposed between the second portion body 234 and the mover 233. The connector 238 may include a first connector portion 238a connected to the second portion body 234 (through the second connecting body 130), a third connector portion 238c connected to the mover 233, and a second connector portion 238b disposed between the first connector portion 238a and the third connector portion 238c. The second connector portion 238b may be rotationally connected to the first connector portion 238a and the third connector portion 238c.

In some examples, the mover 233 may be in the form of a threated rod, and the third connector portion 238c may include a threaded hole configured to receive the mover 233. In some examples, when the mover 233 is rotated in one direction (e.g., counterclockwise), for example, using the graspable device 236, the third connector portion 238c may move upward (e.g., in a plantar to dorsal direction), which may move the first connector portion 238a away from the mover 233/third connector portion 238c, thereby rotating the second portion body 234 in the second direction 235 (e.g., in a dorsal to plantar direction), for example, around the longitudinal axis 125 of the rotating rod 124.

When the second moving mechanism 232 is rotated in another direction (e.g., clockwise), the third connector portion 238c may move downward (e.g., in a dorsal to plantar direction), which may move the first connector portion 238a toward the mover 233/third connector portion 238c, thereby rotating the second portion body 234 in the second direction 235 (e.g., in a plantar to dorsal direction). Other configurations/features/characteristics of the main correction assembly 100 of FIGS. 10A and 10B may be similar to and/or same as the ones described above with respect to the main correction assembly 100 illustrated with respect to FIGS. 1 to 6 and, thus, duplicate description may be omitted.

In some examples, a method for controlled realignment of a bone (e.g., first metatarsal bone) in multiple planes for a Lapidus surgery using the system 10 disclosed herein is provided. The steps presented herein may be performed in any suitable order and combination, and may be modified by or combined with any of the other procedures and features disclosed elsewhere herein.

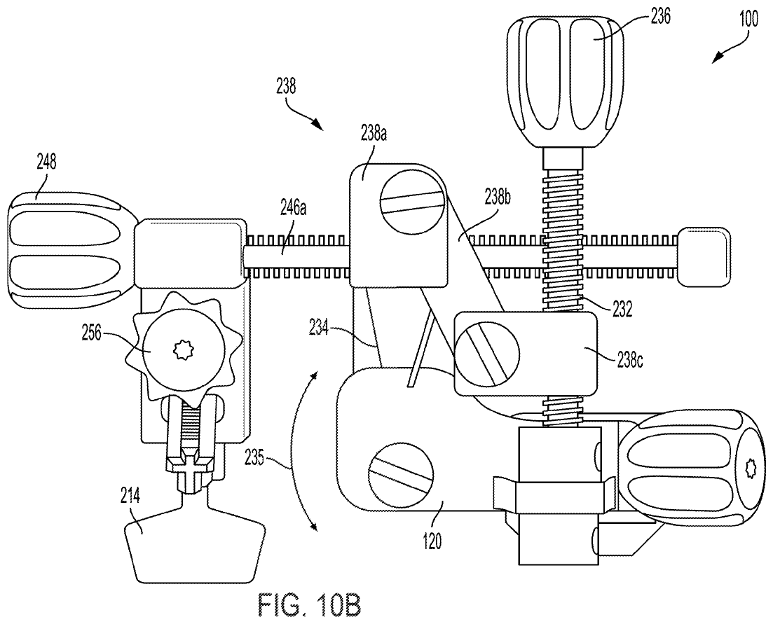

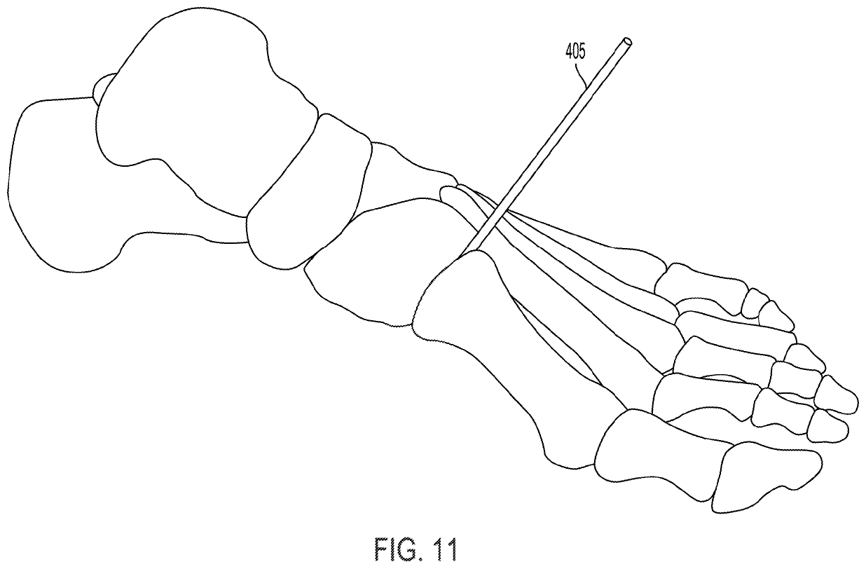

The method may include, as shown in FIG. 11, inserting a first bone fastener 405 into a joint (e.g., first tarsometatarsal joint) adjacent the second bone of the patient, for example, in a dorsal to plantar direction. After the first bone fastener 405 is inserted into the joint, the main correction assembly 100 may be aligned with respect to the first and second bones of the patient. For example, the aligning of the main correction assembly 100 may include attaching the alignment guide 300 to the main correction assembly 100 (e.g., the fixation body 110), and inserting the first bone fastener 405 down through the alignment channel 302, as shown in FIG. 12. Then, the position of the main correction assembly 100 may be adjusted (e.g., rotated around the first bone fastener 405) so that the channels 116a, 116b, 116c of the fixation body 110 may be positioned directly over the first bone. The securing device 340 of the alignment guide 300 may be tightened to secure the position.

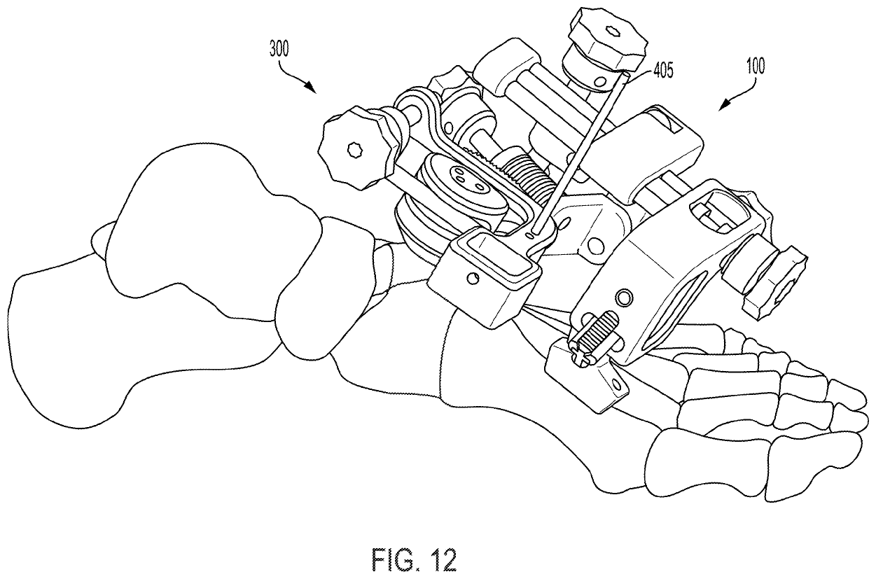

Then, the fixation body 110 may be fixed to the first bone of the patient as shown in FIG. 13. For example, one or more bone fasteners 410, 415 may pass through the one or more channels 116a, 116b, 116c of the fixation body 110 and be inserted into the first bone, for example, percutaneously.

In some examples, after the fixing body 110 is fixed to the first bone, the first bone fastener 405 may be removed by pulling the first bone fastener 405 out of the joint and the alignment channel 302. The alignment guide 300 may be also removed by detaching the alignment guide 300 from the main correction assembly 100. In some examples, after the fixing body 110 is fixed to the first bone, the main correction assembly 100 may be adjusted so that the channels 216a, 216b of the anchor base may be positioned directly over the second bone (e.g., distal portion of the shaft of the second bone).

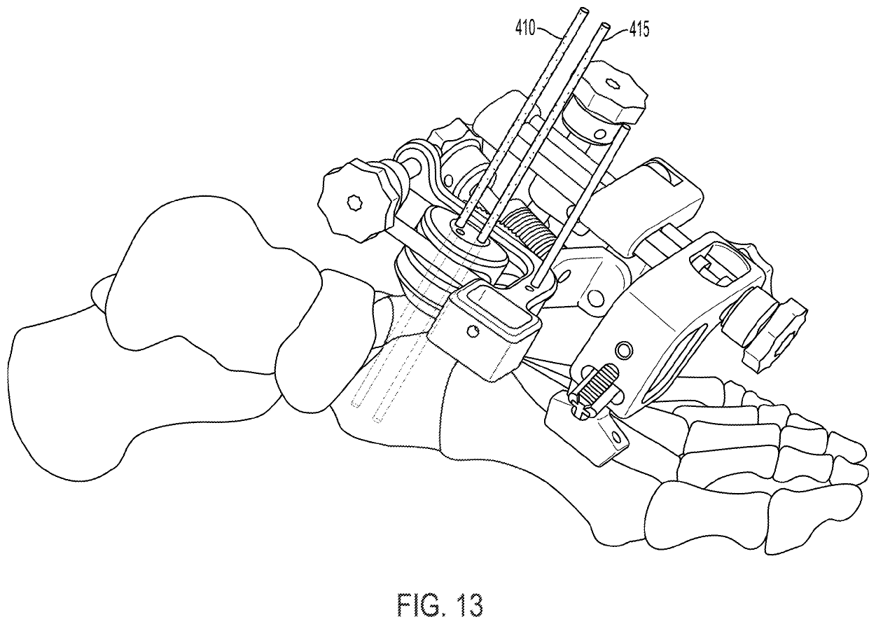

Then, the anchor body 210 may be fixed to the second bone of the patient as shown in FIG. 14. For example, one or more bone fasteners 420, 425 may pass through the one or more channels 216a, 216b of the anchor body 210 and be inserted into the second bone, for example, percutaneously.

Then, the anchor body 210 may be moved/rotated in the first, second, and/or third directions 225, 235, 245 to correct the position of the second bone in the first, second, and/or third planes. In some examples, the anchor body 210 may be moved/rotated, using the fourth moving mechanism 252 of the fourth portion 250, relative to the fixation body 110 in the fourth direction 255 in the third (frontal) plane to correct the position of the second bone in the third plane.

If the first metatarsal bone is not corrected in the frontal plane, there could be a bone misalignment issue. For example, there are two little bones underneath the first metatarsal bone, called a sesamoid bone, and if the metatarsal bone is corrected only in the transverse plane, these bones may get out of the alignment, which may cause a significant unbalance in the soft tissue down around the toes. This may also cause various other bunion-associated problems. Aspects of the present disclosure may provide a way to correct the frontal plane rotation, for example, using the fourth moving mechanism 252, which may ensure that the sesamoid bones are back under the joint with the phalanges.

Then, the anchor body 210 may be moved/rotated, using the first moving mechanism 222, relative to the fixation body 110 in the first direction 225 in the first (transverse) plane to correct a position of the second bone in the first plane. After the first plane correction of the second bone, the anchor body 210 may be moved/rotated, using the second moving mechanism 232 of the second portion 230 relative to the fixation body 110 in the second direction 235 in the second (sagittal) plane to correct the position of the second bone in the second plane. Although it is described that the second bone is corrected in the order of third plane-first plane-second plane above, the second bone can be corrected in any other suitable order (e.g., third plane-second plane-first plane).

In some examples, before, after, and/or while the second bone is corrected in the first, second, and/or third plane, the joint between the second bone and the third bone may be distracted, if necessary, to reposition the second bone at a desired position.

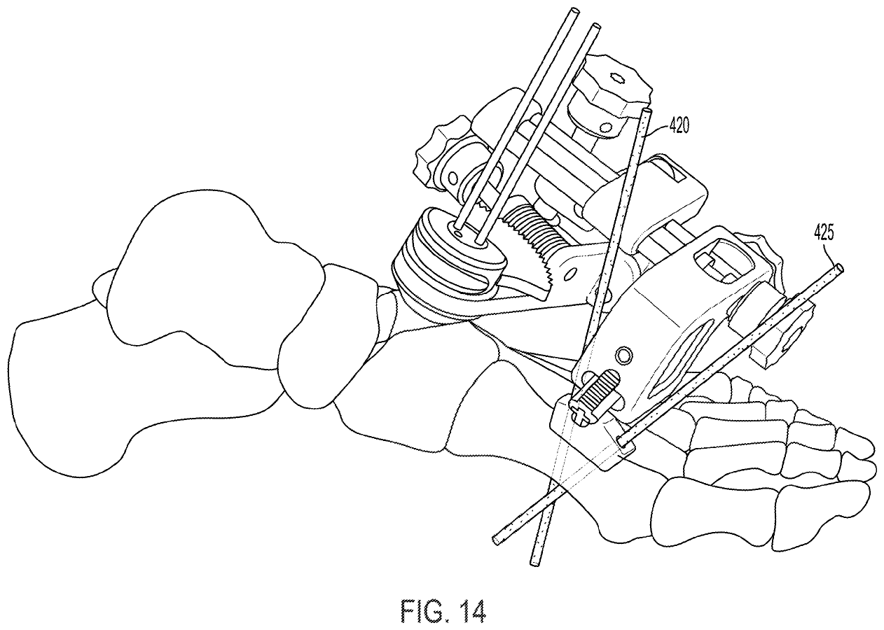

After the second bone is corrected in the first, second, and/or third plane, the alignment guide 300 may be reattached to the fixation body 110 as shown in FIG. 15. The cutting guide 360 may also be inserted into the receiver 350 of the alignment guide 300.

Then, the second bone may be cut by a bone cutting device. The bone cutting device may be inserted into the second bone along the cutting guide 360, for example, by inserting the bone cutting device into the second bone through a first cutting slot 365a thereof. In some examples, the third bone may be cut by inserting the bone cutting device into the third bone along the cutting guide 360. For example, the bone cutting device may be inserted into the third bone through a second cutting slot 365b. In some examples, the cutting guide 360 may include one or more guide surfaces 367a, 367b (see FIG. 9), and in this case, the bone cutting device may be abutted adjacent to the guiding surfaces 367a, 367b to cut the second/third bone.

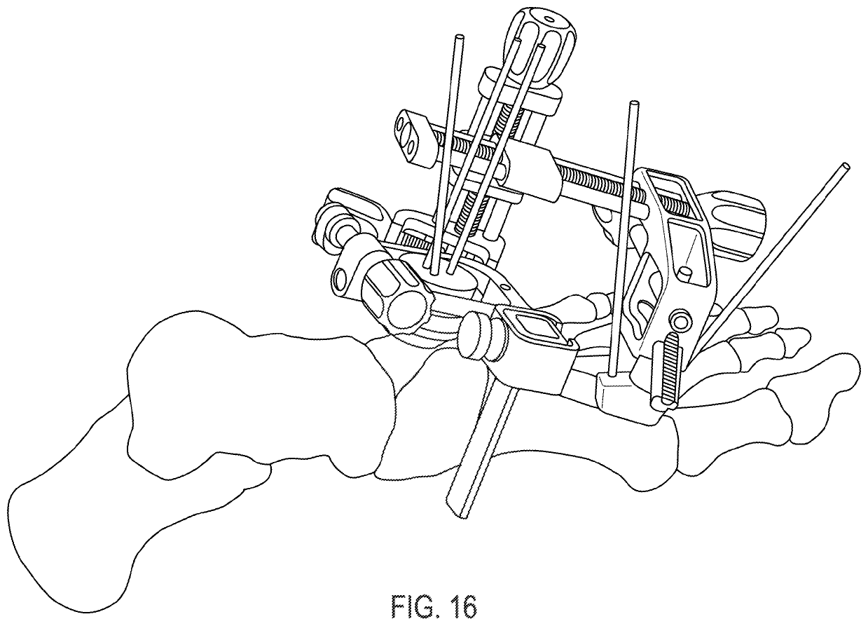

FIG. 17 is a diagram of a top view of an anatomical structure of a foot of a patient with a bunion. As shown in FIG. 17, a first incision 505 may be formed near a joint between the second bone 520 (e.g., a first metatarsal bone) and the third bone 530 (e.g., medial cuneiform bone), for example, on a side of the foot. In some examples, a first cut may be made along the line 532, for example, over the second bone 520 (e.g., first metatarsal bone).

In some examples, a second cut may be made along the line 534 to cut the third bone 530. In some examples, the first cut line 532 may be substantially parallel to the second cut line 534. In some examples, the first cut line 532 and the second cut line 534 may be substantially parallel to a joint line between the second bone 520 and the third bone 530. In some examples, the first cut line 532 and the second cut line 534 may be substantially perpendicular to the longitudinal axis of the second bone. In some examples, the first and second cut may be intended to cut the surface on the second and third bones 520, 530 down to the bloody bone surface to prepare the first and second bones 520, 530 for fusion.

In some examples, the size of the first incision 505 may be in a range of about 3 mm to about 10 mm, for example, about 3 mm to about 5 mm, about 5 mm to about 7 mm, about 7 mm to about 9 mm, and/or about 9 mm to about 10 mm. In other examples, the first incision 505 may have any other suitable size (less than 3 mm or greater than 10 mm). In some examples, a separate incision may be provided for each cut (e.g., one incision for the first cut and another incision for the second cut).

In some examples, one or more additional incisions are provided. For example, a second incision 510 may be provided on the dorsal/top side of the foot. The surgeon may run irrigation through the second incision 510 to clear out debris and any removed materials. The size of the second incision 510 may be the same as or similar to the size of the first incision 505. The second incision 510 may be provided also to protect soft tissues that should not be cut/removed (e.g., blood vessels, tendons, muscles) during the first/second cuts. For example, a blocking device may be provided through the second incision 510 to prevent the bone cutting device (e.g., a bur) from cutting the soft tissues that need to be protected.

In some examples, prior to correcting the second bone in the first, second, and/or the third plane, a soft tissue release may be performed, through the incision 505 on the medial side of the joint between the second bone 520 and the third bone 530, to free up the joint. The soft tissue to be released may include ligaments that may hold the second and third bones together. Some of this soft tissue may be cut or removed so that the second bone 520 can be moved around to correct the angle/position thereof. This soft tissue release may detach the second bone 520 from the third bone 530 and free up the space between these bones to prepare these two bones for fusion. The soft tissue release can be performed through a scalpel or any other suitable sharp device. After the soft tissue release, the connection between the second bone 520 and the third bone 530 may become loose, but to some extent it may still be held together though muscles and tendons. In some examples, a distraction device may be used to loosen the connection between the second and third bones.

Once the joint between the second bone and the third bone is freed up through the soft tissue release prior to the correction of the second bone in the first, second, and/or third plane, the first metatarsal bone and the medial cuneiform bone may not be stable. The system according to the present disclosure may be able to reposition the first metatarsal bone relative to the intermediate cuneiform (or other stable bone), which may be the basis for measuring the intermetatarsal angle and this may avoid any instability or misalignment of the medial cuneiform while also avoiding stressing and potentially breaking the second metatarsal bone (or other metatarsal bones).

In some examples, a portion of the second bone 520 and the third bone 530 that are cut by the bone cutting device may be removed, for example, through the incision 505. In some examples, after the second and/or the third bone is cut, the anchor body 210 may be moved, using the third moving mechanism 242 of the third portion 240, relative to the fixation body 110 in the third direction 245. The movement of the anchor body 210 relative to the fixation body 110 in the third direction 245 may cause the joint (e.g., first tarsometatarsal joint) adjacent the second bone to be compressed or distracted.

In some examples, after the cutting of the second and third bones, the joint may be compressed using the third moving mechanism 242. In this way, the second bone may be pushed close to the third bone, and fused together (e.g., the first metatarsal screwed or plated to the medial cuneiform) so that those two bones are brought together to form one new bone without a joint. The second and third bones may be fixed to each other by any suitable means (e.g., using plates, screws, etc.). Then, the (human) body may initiate a healing process, which may grow the two bones together.

In some examples, after the cutting of the second and third bones (before the compression of the joint), the joint between the second bone and the third bone may be distracted to prepare the bone surfaces for fusion by fenestration or any other suitable means.

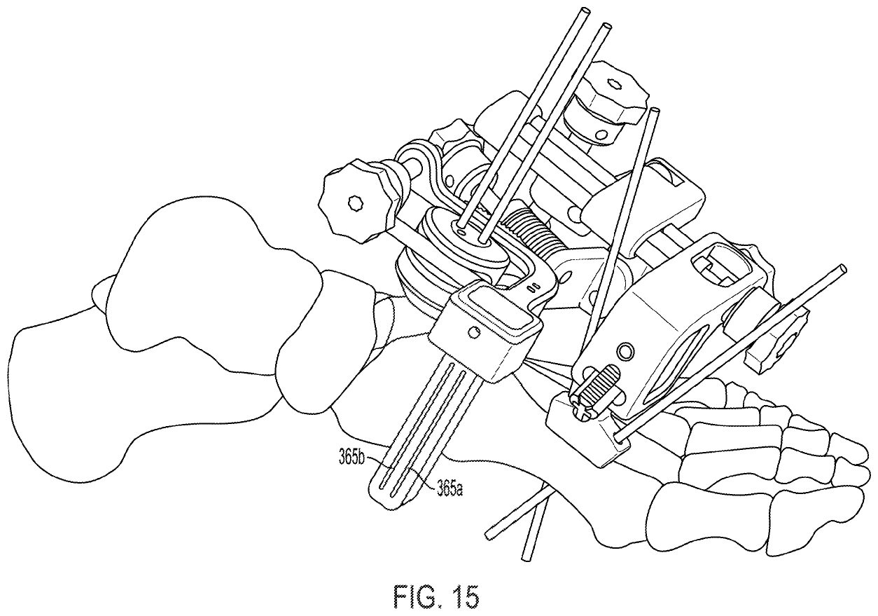

Referring to FIG. 16, in some examples, aligning the main correction assembly may include attaching the alignment guide 300 to the fixation body 110, inserting a joint alignment guide (e.g., cutting guide 360, which may be shown in FIGS. 9 and 16) in the receiver 350 of the alignment guide 300, and aligning a ridge (e.g., side edge 368a/368b) of the joint alignment guide 360 with a joint adjacent to the second bone. In some examples, after the fixation body 110 is fixed to the first bone, the alignment guide 300 and the joint alignment guide 360 may be removed by detaching the alignment guide 300 from the main correction assembly 100.

Aspects of the present disclosure may guide a surgeon in making osteotomies using a bone cutting device through a small incision for a minimally invasive surgery (MIS), particularly for a multi-planar Lapidus procedure. The system 10 according to the present disclosure allows the surgeon to make two (parallel) cuts near a joint (e.g., first tarsometatarsal joint) through the small incision (e.g., around 3 mm to 3.5 mm), which is smaller than the traditional approaches (e.g., having an approximately 3.5 cm incision), thereby having less scarring, shorter soft tissue healing times, and lower risk of infection compared to the traditional full exposure techniques. In some examples, aspects of the present disclosure can be applied to other midfoot fusions (metatarsal adductus) and carpometacarpal joint fusions of the hand.

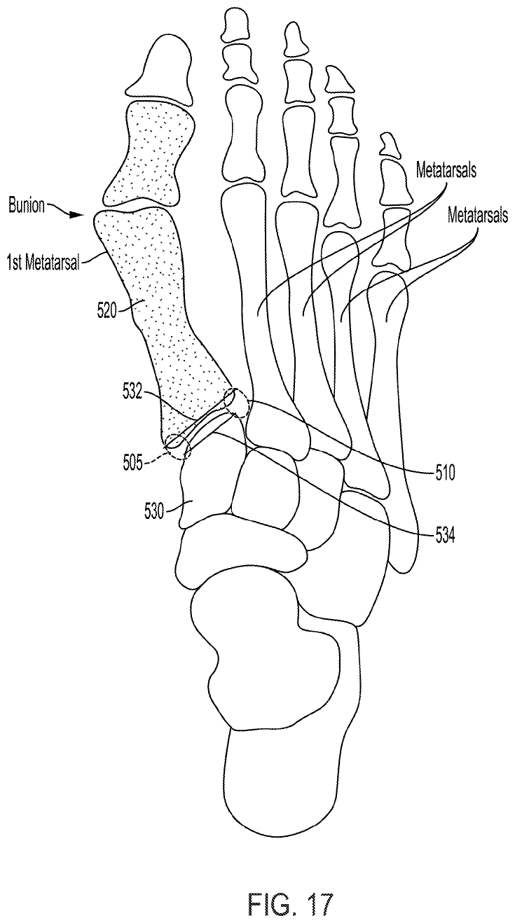

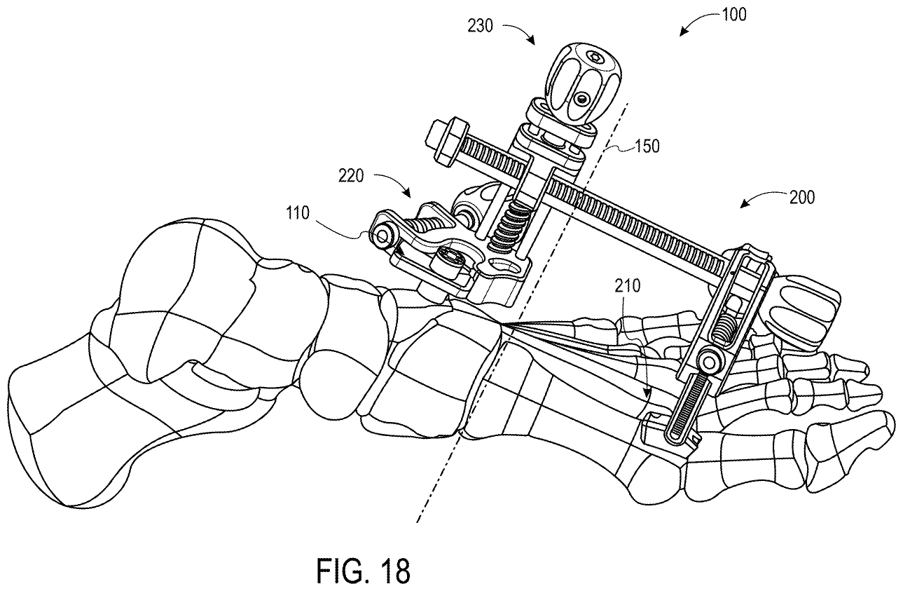

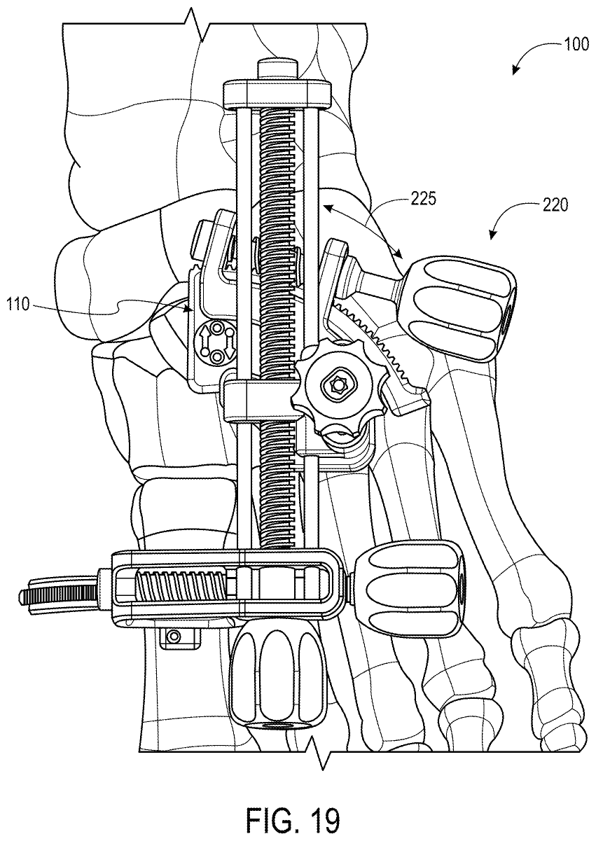

FIGS. 18-20 illustrate another example of a system for controlled realignment of a bone in multiple planes according to another example of the present disclosure. The system may include a main correction assembly 100. In some examples, the main correction assembly 100 may include a fixation body 110 configured to be fixed to a first bone of a patient and a movable body 200 configured to move relative to the fixation body 110. The movable body 200 includes an anchor body 210 configured to be fixed to a second bone of the patient, and a first portion 220 configured to rotate the anchor body 210 relative to the fixation body in a first rotating direction 225 in a first plane. In some examples, the first plane may be a transverse plane.

In some examples, the main correction assembly 100 may define a rotation axis 150 about which the movable body 200 (e.g., anchor body 210 and/or the first portion 220) may be rotated relative to the fixation body 110 in the first plane. In some examples, the main correction assembly 100 may be configured such that the rotation axis 150 is disposed substantially within a joint adjacent the second bone when the fixation body is fixed to the first bone. For example, the rotation axis 150 or at least a portion thereof may be disposed within the joint adjacent the second bone, or a shortest distance between the rotation axis 150 and the joint may be less than about 5 mm, preferably, less about 3 mm, more preferably, less than 1 mm.

In some examples, the first bone may be an intermediate cuneiform bone. In other examples, the first bone may be a lateral cuneiform bone, a cuboid bone, a navicular bone, or any other suitable and/or stable bone of a patient. In some examples, the second bone may be a first metatarsal bone. In other examples, the second bone may be any other bone (e.g., any one of other metatarsal bones) that needs to be corrected. In some examples, the joint adjacent the second bone may be a first tarsometatarsal joint between a medial cuneiform bone and a first metatarsal bone. In other examples, the joint adjacent the second bone may be any one of other tarsometatarsal joints.

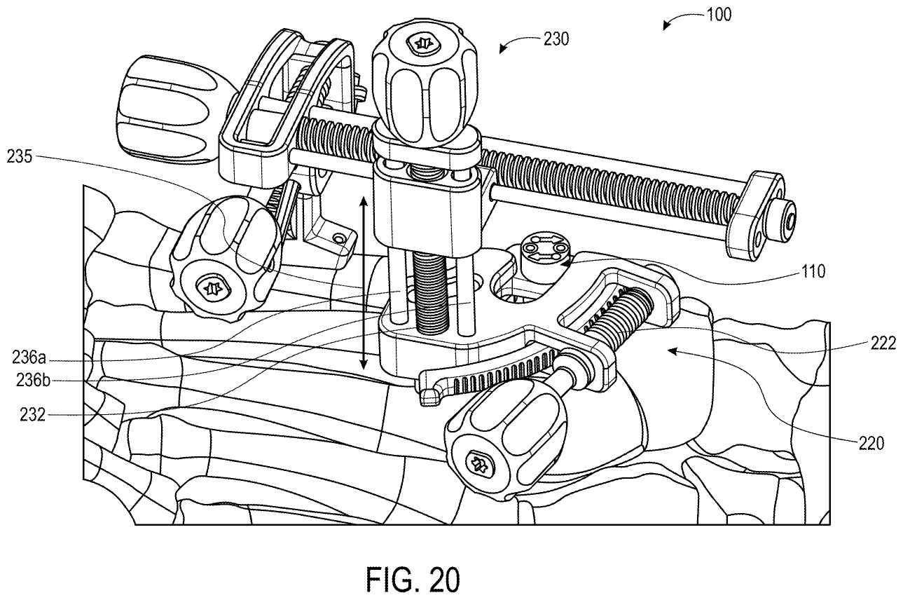

FIG. 21 illustrates an example fixation body 110 of the system of FIG. 18. As shown in FIG. 21, the fixation body 110 may include a main body 161 and a first curved arm 162. The first curved arm 162 may extend from the main body 161. For example, the first curved arm 162 may extend from an upper side portion of the main body 161. In some examples, the first portion 220 of the movable body 200 may be configured to rotate along the curved arm 162 of the fixation body 110.

In some examples, the fixation body 110 may further include a second curved arm 163. The second curved arm 163 may extend from the main body 161. For example, the second curved arm 163 may extend from an lower side portion of the main body 161. In some examples, the first portion 220 of the movable body 200 may be configured to rotate along both of the first and second curved arms 162, 163 of the fixation body 110. In some examples, the first curved arm 162 and/or the second curved arm 163 may be in a shape of a partial circle.

In some examples, the first curved arm 162 and/or the second curved arm 163 may be configured so that a center C2 of the curve or partial circle of the first curved arm 162 and/or the second curved arm 163 may be disposed substantially within the joint adjacent the second bone when the fixation body 110 is fixed to the first bone. The center C2 of the curve or partial circle may be located within the rotation axis 150. In some examples, the radius R1 of curvature of the first curved arm 162 may be in a range of about 2 cm to about 7 cm, more preferably in a range of about 3 cm to about 5 cm.

When the rotation axis 150 is not located substantially within the joint adjacent the second bone, the proximal end of the first metatarsal can be pushed into the second metatarsal while the movable body 200 is rotated relative to the fixation body 110, thereby causing an impingement issue. Aspects of the present disclosure may address the impingement issue by configuring the main correction assembly 100 to have the rotation axis 150 substantially within the joint adjacent the second bone when the main correction assembly 100 is fixed to the foot of the patient.

In some examples, the first curved arm 162 of the fixation body 110 may be engaged with the first moving mechanism 222 of the first portion 220. For example, the first moving mechanism 222 may be rotationally coupled to the first curved arm 162 of the fixation body 110. In some examples, the first curved arm 162 and the first moving mechanism 222 together may form a gear arrangement. For example, the first moving mechanism 222 may be in the form of a worm/threaded rod/screw, and the first curved arm 162 may include teeth 164. In other examples, the first curved arm 162 and the first moving mechanism 222 together may form any other suitable arrangement/adjustment mechanism, through which the anchor body 210 may rotate relative to the fixation body 110 in the first direction 225 in the first plane.

The first curved arm 162 may have a free end portion 165. The first curved arm 162 may have a stopper 167 at the free end portion 165. The stopper 167 may protrude from the free end portion 165 of the first curved arm 162. The stopper 167 may be provided to prevent the first curved arm 162 (and ultimately the fixation body 110) from being detached/disassembled from the first portion 220. In some examples, the second curved arm 163 may have a stopper/protrusion at a free end portion thereof to prevent the second curved arm 162 (and ultimately the fixation body 110) from being detached/disassembled from the first portion 220.

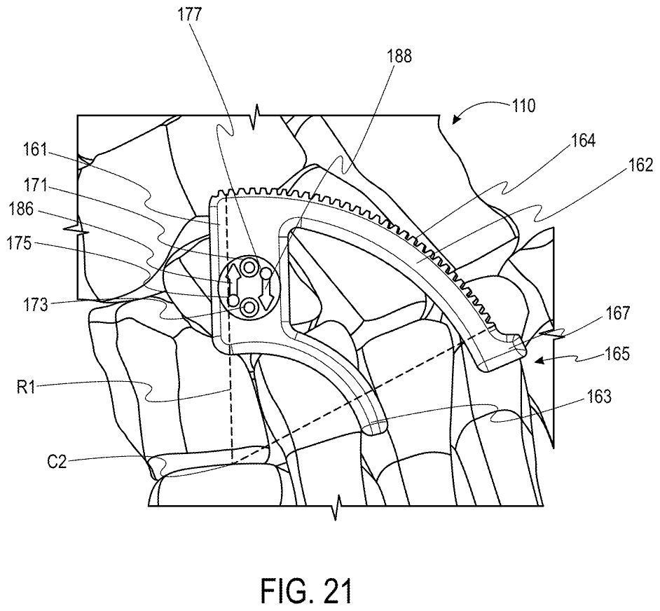

Referring to FIGS. 21, 22A-C, and 23, in some examples, the fixation body 110 may include one or more channels configured to receive a bone fastener to (temporally or removably) fix the fixation body 110 to the first bone. The one or more channels of the fixation body 110 may include a first channel 171 having a first trajectory 172 and a second channel 173 having a second trajectory 174. In some examples, the first and second trajectories 172, 174 may be parallel to each other. In this way, after the fixation body 110 is fixed to the first bone, the main correction assembly 100 can still be moved along the first/second trajectory 172/174 (e.g., in a dorsal to plantar or plantar to dorsal direction). In some examples, the first and second trajectories 172, 174 may be substantially orthogonal to the first plane.

In some examples, the one or more channels of the fixation body 110 may include a third channel 175 having a third trajectory 176. The third trajectory 176 may be non-parallel to the first and second trajectories 172, 174. Therefore, when bone fasteners are inserted into the third channel 175 and at least one of the first and second channels 171, 173, the movement of the main correction assembly 100 along the first/second trajectory 172/174 (e.g., in a dorsal to plantar or plantar to dorsal direction) may be prevented. The angle between the first/second trajectory 172/174 and the third trajectory 176 may be in a range of about 5° to about 25°. In other examples, the angle between the first/second trajectory 172/174 and the third trajectory 176 may have any other suitable value (less than 5° or greater than 25°).

In some examples, the one or more channels of the fixation body 110 may include a fourth channel 177 having a fourth trajectory 178. The fourth trajectory 178 may be non-parallel to the first and second trajectories 172, 174. Therefore, when bone fasteners are inserted into the fourth channel 177 and at least one of the first and second channels 171, 173, the movement of the main correction assembly 100 along the first/second trajectory 172/174 (e.g., in a dorsal to plantar or plantar to dorsal direction) may be prevented. The angle between the first/second trajectory 172/174 and the fourth trajectory 178 may be in a range of about 5° to about 25°. In other examples, the angle between the first/second trajectory 172/174 and the fourth trajectory 178 may have any other suitable value (less than 5° or greater than 25°).

In some examples, the fourth trajectory 178 may be non-parallel to the third trajectory 176. In some examples, the third and fourth trajectories 176, 178 are symmetrical to each other relative to an axis that is parallel to the first and second trajectories. In some examples, the diameter of the bone fastener may be smaller than the diameter of the one or more channels.

In some examples, the fixation body 110 may include one or more visual markers 186, 188 indicating a direction of the third and/or fourth trajectories (e.g., direction of the channel from the opening in the top view). For example, the fixation body 110 may include a first marker 186 indicating a direction of the third trajectory 176, and a second marker 188 indicating a direction of the fourth trajectory 178.

In some examples, the movable body 200 may include a second portion 230 movably connected to the first portion 220. The second portion 230 may have a second moving mechanism 232. The second moving mechanism 232 may be configured to move the anchor body 210 relative to the fixation body 110 in the second direction 235 in the second plane.

In some examples, the second moving mechanism 232 may further move all components disposed between the second moving mechanism 232 and the anchor body 210 in the second direction 235 in the second plane. In some examples, the second plane may be perpendicular to the first plane. The second plane may be a sagittal (dorsal/plantar) plane or substantially parallel to the sagittal plane (for example, when the main correction assembly 100 is attached to a human body). In this example, the second direction 235 may be a dorsal to plantar or plantar to dorsal direction.

In some examples, the first portion 220 may include a hole configured to receive the second moving mechanism 232. The first portion 220 and the second moving mechanism 232 may have a threaded engagement. For example, the second moving mechanism 232 may include a thread, and the hole of the first portion 220 may include an internal thread. The rotation of the second moving mechanism 232 may move the second moving mechanism 232 (and ultimately the second portion 230) relative to the first portion 220 in the second direction 235.

In some examples, the second moving mechanism 232 may be in a shape of a rod (e.g., threaded rod). In other examples, the second moving mechanism 232 may have any other suitable shape. In some examples, the second portion 230 may further include one or more support bodies 236a and 236b that may extend in parallel with the second moving mechanism 232. In this case, the first portion 220 may further include one or more holes configured to receive the one or more support bodies 236a and 236b.

In other examples, the second moving mechanism 232 may move up and down (in the second direction 235) in any other suitable manner (e.g., simply pushing or pulling the moving mechanism 232 up and down).

Other configurations/features/characteristics of the main correction assembly 100 of FIG. 18 may be similar to and/or same as the ones described above with respect to the main correction assembly 100 illustrated with respect to FIGS. 1 and/or 10A and, thus, duplicate description may be omitted.

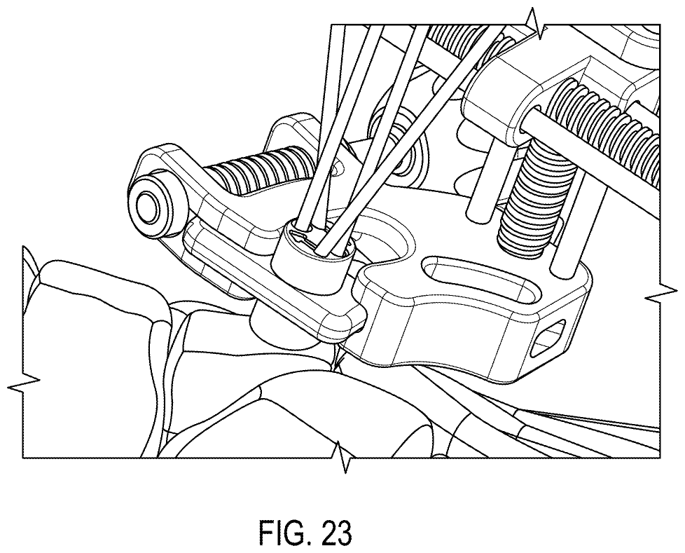

FIG. 24 illustrates a system for controlled realignment of a bone in multiple planes according to another example of the present disclosure. The system may include a main correction assembly 100 and a cutting guide assembly (CGA) 600.

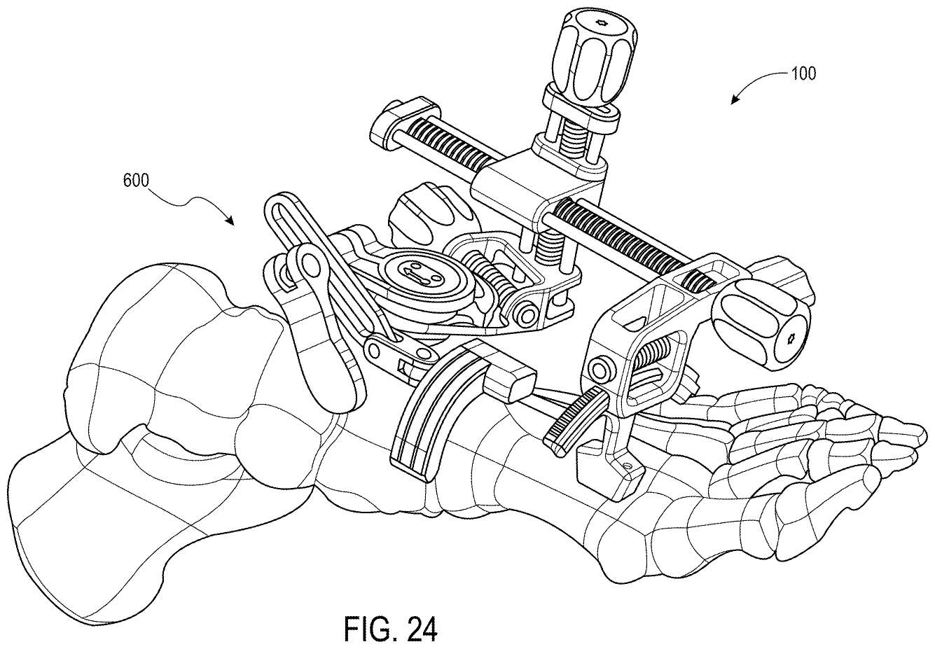

As shown in FIG. 25, the main correction assembly 100 may include a fixation body 110 configured to be fixed to a first bone of a patient and a movable body 200 configured to move relative to the fixation body 100. Other configurations/features/characteristics of the main correction assembly 100 of FIGS. 24-25 may be similar to and/or same as the ones described above with respect to the main correction assembly 100 illustrated with respect to FIGS. 1, 10A, and/or 18 and, thus, duplicate description may be omitted.

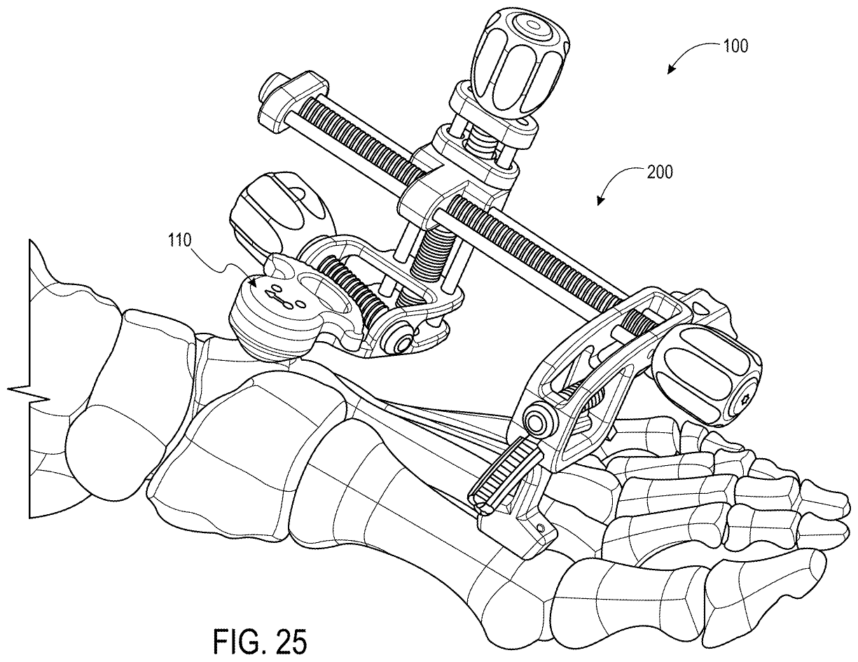

Referring to FIG. 26, the cutting guide assembly 600 may include a first CGA portion 610 configured to be removably coupled to the fixation body 110 of the main correction assembly 100, a second CGA portion 620, and a cutting guide 630 configured to guide a bone cutting device. The first CGA portion 610 may be movably coupled to the second CGA portion 620.

The first CGA portion 610 may include an opening 611. In some examples, an inner surface 612 of the first CGA portion 610 within the opening 611 is configured to secure to an outer surface of the fixation body 110. In some examples, the first CGA portion 610 may be in a ring or hoop shape as shown in FIG. 26. In other examples, the first CGA portion 610 may have any other suitable shape (e.g., elliptical, rectangular, triangle, square, hexagon, or any other suitable polygonal shape). In some examples, the first CGA portion 610 may be rotatable relative to the second CGA portion 620 about the rotating axis 614 in a rotating direction 615.

The second CGA portion 620 may be movably coupled to the cutting guide 630. In some examples, the second CGA portion 620 may be an (elongated) arm. The arm may include a slot 622. The slot 622 may be elongated. In some examples, the first CGA portion 610 may be movable relative to the second CGA portion 620 along the slot 622, for example, in a fifth direction 616.

In some examples, the second CGA portion 620 may be configured to move/rotate relative to the cutting guide 630 in a sixth direction 625. In some examples, the fifth direction 616 may be (substantially) perpendicular to the sixth direction 625.

The cutting guide 630 may include one or more cutting slots 631a, 631b configured to receive the bone cutting device. In some examples, the one or more cutting slots may include a first cutting slot 631a and a second cutting slot 631b. The first cutting slot 631a may be provided to guide the bone cutting device to cut the third bone, and the second cutting slot 631b may be provided to guide the bone cutting device to cut the second bone. Although only two cutting slots are shown in FIG. 26, more or less than two cutting slots (e.g., 1, 3, 4, 5, . . . ) can be provided with the cutting guide 630.

In some examples, the first cutting slot 631a may be in parallel with the second cutting slot 631b. In other examples, the first cutting slot 631a may be not parallel to the second cutting slot 631b. In some examples, as shown in FIG. 26, at least a portion of the cutting guide 630 and the one or more cutting slots may be curved. In other examples, the entire body of the cutting guide 630 may be in a flat shape.

In some examples, the cutting guide 630 may include one or more distal channels 633 configured to receive a bone fastener to fix the cutting guide 630 to the second bone. In some examples, the cutting guide 630 may include a plurality of distal channels 633, and at least two of the distal channels 633 may have a different diameter for bone fasteners in different sizes.

In some examples, the cutting guide 630 may also include one or more central channels 634 configured to receive a bone fastener to be inserted into a joint adjacent the second bone to align the cutting guide 630 with the joint. In some examples, the one or more central channels 634 may be disposed between the first cutting slot 631a and the second cutting slot 631b. In some examples, the cutting guide 630 may include a plurality of central channels 634, and at least two of the central channels 634 may have a different diameter for bone fasteners in different sizes.

In some examples, the one or more central channels 634 may serve as an alignment channel similar to the alignment channel 302 of the alignment guide 300. That is, the cutting guide assembly 600 and the main correction assembly 100 may be aligned with respect to the first bone and the second bone using the one or more central channels 634 and the bone fastener inserted into the one or more central channels 634. For example, the bone fastener may be inserted into a joint (e.g., first tarsometatarsal joint) adjacent the second bone of the patient, and the bone fastener may be inserted down through the one or more central channels 634 while the cutting guide assembly 600 is coupled to the main correction assembly 100.

In some examples, the cutting guide assembly 600 may further include an adjustable fastening mechanism 640. The adjustable fastening mechanism 640 may be configured to fasten and unfasten the first CGA portion 610 to and from the second CGA portion 620. The fastening of the first CGA portion 610 to the second CGA portion 620 by the adjustable fastening mechanism 640 may prevent the first CGA portion 610 from moving and/or rotating relative to the second CGA portion 620. In some examples, the adjustable fastening mechanism 640 may be a cam lock lever. In other examples, the adjustable fastening mechanism 640 may be any other suitable fastening means.

In some examples, the cutting guide assembly 600 may further include a connector 650. The connector 650 may connect the second CGA portion 620 with the cutting guide 630. In some examples, the cutting guide 630 may be configured to move relative to the connector 650 in a seventh direction 635. In some examples, the seventh direction 635 may be (substantially) perpendicular to the sixth direction 625. In some examples, the plane where the seventh direction 635 is disposed may be (substantially) perpendicular to the plane where the sixth direction 625 is disposed. In some examples, the fifth direction 616 may be disposed within the plane where the sixth direction 625 is disposed.

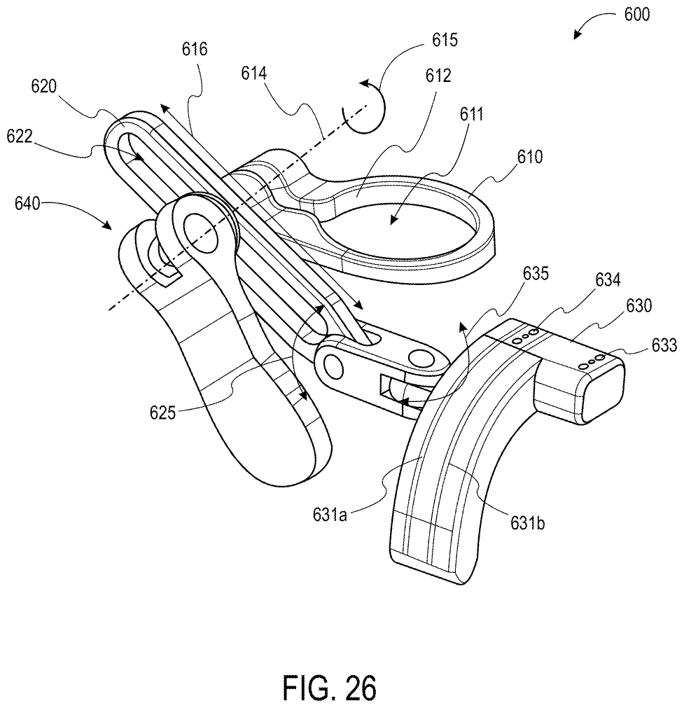

FIG. 27 illustrates an example cutting guide 700 according to an example of the present disclosure. The cutting guide 700 may include one or more cutting slots configured to receive a bone cutting device. In some examples, the one or more cutting slots may include a first cutting slot 710a and a second cutting slot 710b. The first cutting slot 710a may be provided to guide the bone cutting device to cut the third bone, and the second cutting slot 710b may be provided to guide the bone cutting device to cut the second bone. Although only two cutting slots are shown in FIG. 27, more or less than two cutting slots (e.g., 1, 3, 4, 5, . . . ) can be provided with the cutting guide 700. In some examples, the first cutting slot 710a may be in parallel with the second cutting slot 710b. In other examples, the first cutting slot 710a may be not parallel to the second cutting slot 710b. In some examples, the entire body of the cutting guide 700 may be in a flat shape.

In some examples, the cutting guide 700 may include one or more fixation channels configured to receive a bone fastener to fix the cutting guide 700 to the second bone and the third bone. For example, the cutting guide 700 may include a proximal fixation channel 722 configured to receive a first bone fastener to fix the cutting guide 700 to the third bone of the patient, and a distal fixation channel 724 configured to receive a second bone fastener to fix the cutting guide to the second bone of the patient.

In some examples, the proximal fixation channel 722 and the distal fixation channel 724 may be disposed in the opposite sides relative to the cutting slots 710a, 710b. In some examples, the proximal fixation channel 722 and the distal fixation channel 724 may define a straight line extending from the proximal fixation channel 722 to the distal fixation channel 724, and the straight line may be substantially perpendicular to at least one of the cutting slots 710a, 710b.

In some examples, the cutting guide 700 may also include a central channel 732 configured to receive a bone fastener to be inserted into a joint adjacent the second bone to align the cutting guide 700 with the joint, the second bone, and/or the third bone. In some examples, the central channel 732 may be disposed between the first cutting slot 710a and the second cutting slot 710b. In some examples, the central channel 732 may be disposed on the straight line defined by the proximal fixation channel 722 and the distal fixation channel 724. Although only one proximal fixation channel 722, one distal fixation channel 724, and one central channel 732 are shown in FIG. 27, each of these channels may have more than one channel (e.g., 2, 3, 4, 5, . . . ).

In some examples, the diameter of the proximal fixation channel 722 and the distal fixation channel 724 may be greater than the diameter of the central channel 732 so that the proximal fixation channel 722 and the distal fixation channel 724 can receive a bone fastener having a greater diameter than the central channel 732. In other examples, the diameter of the proximal fixation channel 722 and the distal fixation channel 724 may be the same as the diameter of the central channel 732.

In some examples, a shortest distance between the proximal fixation channel 722 and the first cutting slot 710a may be in a range of about 2 mm to about 10 mm. Similarly, a shortest distance between the distal fixation channel 724 and the second cutting slot 710b may be in a range of about 2 mm to about 10 mm.

In some examples, the cutting guide 700 may be in a cross shape as shown in FIG. 27. For example, the cutting guide 700 may include a main elongated body 702 along which the cutting slots 710a, 710b may extend and elongated, a proximal protrusion 704 extending from the main elongate body 702 and having the proximal fixation channel 722, and a distal protrusion 705 extending from the main elongate body 702 and having the distal fixation channel 724. The distal protrusion 705 may extend from the main elongated body 702 in a direction opposite the direction that the proximal protrusion 704 extend from the main elongated body 702. In other examples, the cutting guide 700 may have any other suitable shape (e.g., circle, rectangle, or any other suitable polygonal shape).

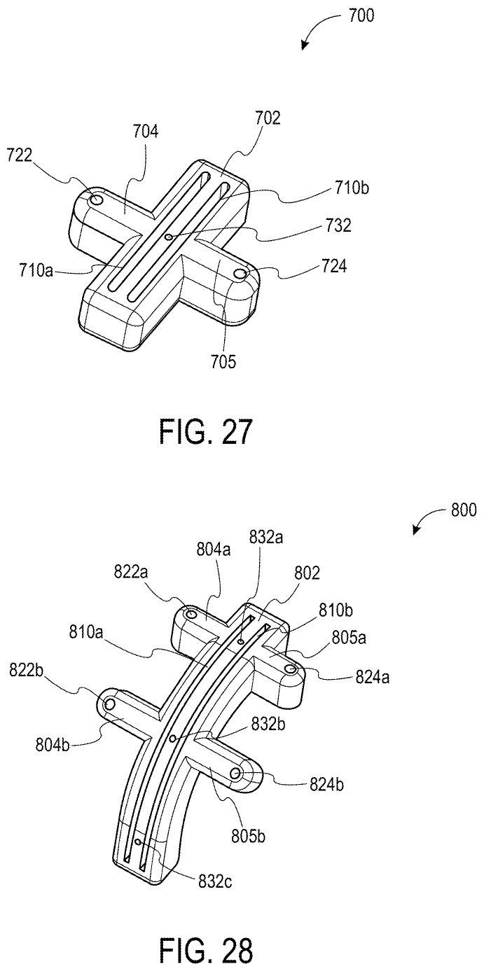

FIG. 28 illustrates another example cutting guide 800 according to an example of the present disclosure. The cutting guide 800 may include one or more cutting slots configured to receive a bone cutting device. In some examples, the one or more cutting slots may include a first cutting slot 810a and a second cutting slot 810b. The first cutting slot 810a may be provided to guide the bone cutting device to cut the third bone, and the second cutting slot 810b may be provided to guide the bone cutting device to cut the second bone. Although only two cutting slots are shown in FIG. 28, more or less than two cutting slots (e.g., 1, 3, 4, 5, . . . ) can be provided with the cutting guide 800. In some examples, the first cutting slot 810a may be in parallel with the second cutting slot 810b. In other examples, the first cutting slot 810a may be not parallel to the second cutting slot 810b. In some examples, the entire portion or at least a portion of the cutting guide 800 and the one or more cutting slots 810a, 810b may be curved.

In some examples, the cutting guide 800 may include one or more fixation channels configured to receive a bone fastener to fix the cutting guide 800 to the second bone and the third bone. For example, the cutting guide 800 may include one or more proximal fixation channels 822a, 822b configured to receive a bone fastener to fix the cutting guide 800 to the third bone of the patient, and one or more distal fixation channels 824a, 824b configured to receive a bone fastener to fix the cutting guide to the second bone of the patient.

In some examples, the one or more proximal fixation channels and the one or more distal fixation channel may be disposed in the opposite sides relative to the cutting slots 810a, 810b. For example, a first proximal fixation channel 822a and a first distal fixation channel 824a may be disposed in the opposite sides relative to the cutting slots 810a, 810b, and a second proximal fixation channel 822b and a second distal fixation channel 824b may be disposed in the opposite sides relative to the cutting slots 810a, 810b.

In some examples, the first/second proximal fixation channel 822a/b and the first/second distal fixation channel 824a/b may define a straight line extending from the first/second proximal fixation channel 822a/b to the first/second distal fixation channel 824a/b, and the straight line may be substantially perpendicular to at least one of the cutting slots 810a, 810b.

In some examples, the cutting guide 800 may also include one or more central channels 832a, 832b, 832c configured to receive a bone fastener to be inserted into a joint adjacent the second bone to align the cutting guide 800 with the joint, the second bone, and/or the third bone. In some examples, the one or more central channels 832a, 832b, 832c may be disposed between the first cutting slot 810a and the second cutting slot 810b. In some examples, the one or more central channels 832a, 832b may be disposed on the straight line defined by the first/second proximal fixation channel 822a/b and the first/second distal fixation channel 824a/b.

In some examples, the diameter of the proximal fixation channels and the distal fixation channels may be greater than the diameter of the one or more central channels so that the proximal fixation channels and the distal fixation channels can receive a bone fastener having a greater diameter than the central channels. In other examples, the diameter of the proximal fixation channels and the distal fixation channels may be the same as the diameter of the central channels.

In some examples, a shortest distance between the one or more proximal fixation channels 822a, 822b and the first cutting slot 810a may be in a range of about 2 mm to about 10 mm. Similarly, a shortest distance between the one or more distal fixation channels 824a, 824b and the second cutting slot 810b may be in a range of about 2 mm to about 10 mm. In some examples, a shortest distance between the first proximal fixation channel 822a and the first cutting slot 810a may be shorter than a shortest distance between the second proximal fixation channel 822b and the first cutting slot 810a. Similarly, a shortest distance between the first distal fixation channel 824a and the second cutting slot 810b may be shorter than a shortest distance between the second distal fixation channel 824b and the second cutting slot 810b.

In some examples, the cutting guide 800 may include multiple proximal and distal protrusions as shown in FIG. 28. The cutting guide 800 may include a main elongated body 802 along which the cutting slots 810a, 810b may extend and elongated. The cutting guide 800 may further include a first proximal protrusion 804a extending from the main elongate body 802 and having the first proximal fixation channel 822a, and a first distal protrusion 805a extending from the main elongate body 802 and having the first distal fixation channel 824a. The cutting guide 800 may also include a second proximal protrusion 804b extending from the main elongate body 802 and having the second proximal fixation channel 822b, and a second distal protrusion 805b extending from the main elongate body 802 and having the second distal fixation channel 824b. The first/second distal protrusion 805a/b may extend from the main elongated body 802 in a direction opposite the direction that the first/second proximal protrusion 804a/b extends from the main elongated body 802. In other examples, the cutting guide 800 may have any other suitable shape (e.g., circle, rectangle, or any other suitable polygonal shape).

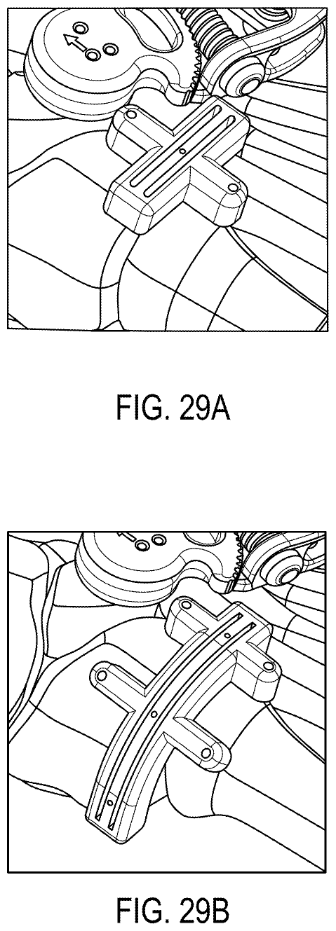

FIGS. 29A and 29B illustrate an example usage of the cutting guides 700, 800 of FIGS. 27 and 28, respectively, according to an example of the present disclosure. Referring to FIG. 29A, the cutting guide 700 may be disposed dorsally around the joint adjacent the second bone so that a bone cutting device can enter dorsally. Referring to FIG. 29B, the cutting guide 800 may cover both the dorsal and medial sides of the joint adjacent the second bone so that a bone cutting device can enter dorsally or dorsomedially. In this way, the cutting guide 700/800 may allow the bone cutting device to be entered from the dorsal or dorsomedial direction, thereby preventing damages to the soft tissues or another bone (e.g., second metatarsal) that can occur when the bone cutting device enters from the medial side.

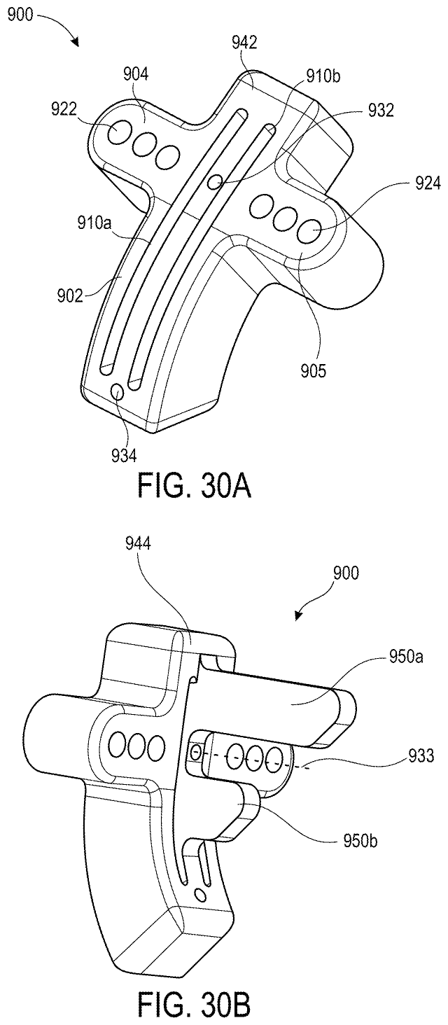

FIGS. 30A and 30B illustrate another example cutting guide 900 according to an example of the present disclosure. The example cutting guide 900 may include one or more cutting slots configured to receive a bone cutting device. In some examples, the one or more cutting slots may include a first cutting slot 910a and a second cutting slot 910b. The first cutting slot 910a may be provided to guide the bone cutting device to cut the third bone, and the second cutting slot 910b may be provided to guide the bone cutting device to cut the second bone. Although only two cutting slots are shown in FIG. 30A, more or less than two cutting slots (e.g., 1, 3, 4, 5, . . . ) can be provided with the cutting guide 900. In some examples, the first cutting slot 910a may be in parallel with the second cutting slot 910b. In other examples, the first cutting slot 910a may be not parallel to the second cutting slot 910b. In some examples, the entire body or at least a portion of the cutting guide 900 and the cutting slots may be curved. In other examples, the entire body of the cutting guide 900 may be in a flat shape.

In some examples, the cutting guide 900 may include one or more fixation channels configured to receive a bone fastener to fix the cutting guide 900 to the second bone and the third bone. For example, the cutting guide 900 may include one or more proximal fixation channels 922 configured to receive a bone fastener to fix the cutting guide 900 to the third bone of the patient, and one or more distal fixation channels 924 configured to receive a bone fastener to fix the cutting guide to the second bone of the patient.

In some examples, the one or more proximal fixation channels 922 and the one or more distal fixation channels 924 may be disposed in the opposite sides relative to the cutting slots 910a, 910b. In some examples, the one or more proximal fixation channels 922 and the one or more distal fixation channels 924 may define a straight line extending from the one or more proximal fixation channel 922 to the one or more distal fixation channels 924, and the straight line may be substantially perpendicular to at least one of the cutting slots 910a, 910b.

In some examples, the cutting guide 900 may be in a cross shape as shown in FIG. 30A. For example, the cutting guide 900 may include a main elongated body 902 along which the cutting slots 910a, 910b may extend and elongated, a proximal protrusion 904 extending from the main elongate body 902 and having the one or more proximal fixation channels 922, and a distal protrusion 905 extending from the main elongate body 702 and having the one or more distal fixation channels 924. The distal protrusion 905 may extend from the main elongated body 902 in a direction opposite the direction that the proximal protrusion 904 extends from the main elongated body 902. In other examples, the cutting guide 900 may have any other suitable shape (e.g., circle, rectangle, or any other suitable polygonal shape).

In some examples, the cutting guide 900 may also include one or more central channels 932, 934 configured to receive a bone fastener to be inserted into a joint adjacent the second bone to align the cutting guide 900 with the joint, the second bone, and/or the third bone. In some examples, a first central channel 932 may be disposed between the first cutting slot 910a and the second cutting slot 910b. In some examples, the central channel 932 may be disposed on the straight line defined by the one or more proximal fixation channels 922 and the one or more distal fixation channels 924. A second central channel 934 may be disposed at a lower portion of the main elongated body 902. In some examples, the first central channel 932 and the second central channel 934 may define a straight line, and the straight line may be parallel with at least one of the cutting slots 910a, 910b.

In some examples, the diameter of the one or more proximal fixation channels 922 and the one or more distal fixation channels 924 may be greater than the diameter of the central channels 932, 934 so that the proximal and distal fixation channels 922, 924 can receive a bone fastener having a greater diameter than the central channels 932, 934. In other examples, the diameter of the proximal and distal fixation channels 922, 924 may be the same as the diameter of the central channels 932, 934.

In some examples, a shortest distance between the innermost proximal fixation channel 922 and the first cutting slot 910a may be in a range of about 2 mm to about 10 mm. Similarly, a shortest distance between the innermost distal fixation channel 924 and the second cutting slot 910b may be in a range of about 2 mm to about 10 mm.

In some examples, the cutting guide 900 may include a top surface 942 and a bottom surface 944 opposite the top surface 942, and the cutting guide 900 may further include one or more fins protruding from the bottom surface 944. The one or more fins may include a first fin 950a and a second fin 950b. The one or more fins may be configured to interact with the joint adjacent the second bone to assist with ensuring the cutting guide 900 is oriented and centered properly relative to the joint.

In some examples, the one or more fins may protrude from the main elongated body 902. The central channel 932 may define a channel center axis 933, and the channel center axis 933 may be disposed between the first fin 950a and the second fin 950b. In some examples, a length of the first fin 950a may be greater than a length of the second fin 950b.

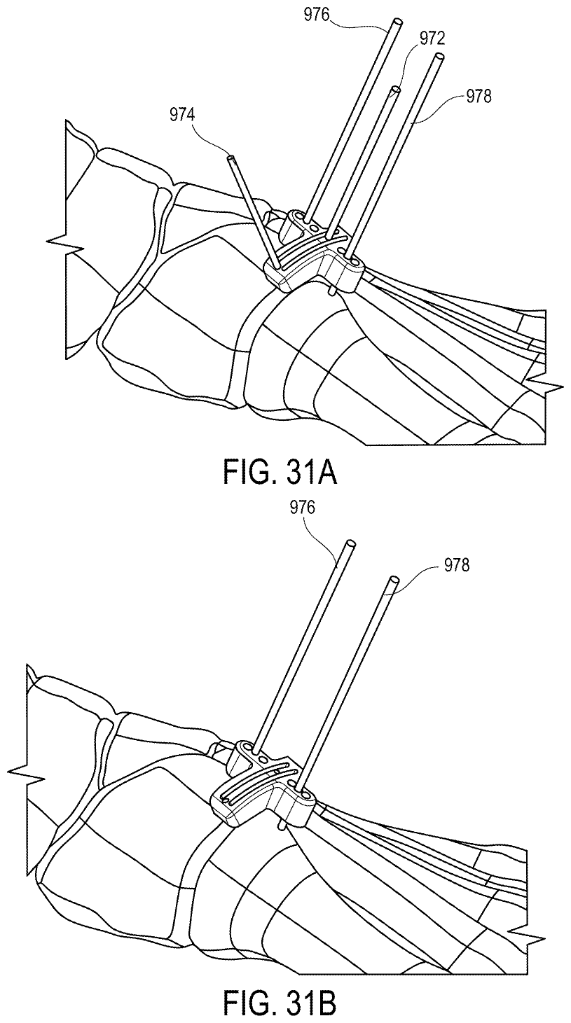

FIGS. 31A and 31B illustrate an example usage of the cutting guide 900 of FIGS. 30A and 30B according to an example of the present disclosure. In some examples, a first bone fastener 972 may be inserted into the joint adjacent the second bone (e.g., first tarsometatarsal joint). The first bone fastener 972 may be coplanar with the joint. Then, the cutting guide 900 may be slid down over the first bone fastener 972 by inserting the first bone fastener 972 down through the central channel 932. Then, in some examples, a second bone fastener 974 may be inserted into the central channel 934 and the joint adjacent the second bone. The second bone fastener 974 may be coplanar with the joint. The use of the second bone fastener 974 may help ensure alignment of the cutting guide 900 relative to the joint and may also more firmly secure the cutting guide 900 while inserting additional bone fasteners in subsequent steps.

After the first bone fastener 972 and/or the second bone fastener 974 are inserted into the joint, third and fourth bone fasteners 976, 978 may be inserted into the proximal and distal fixation channels 922, 924. After the third and fourth bone fasteners 976, 978 are inserted, the first bone fastener 972 and/or the second bone fastener 974 may be removed to create space and/or allow clearance for a bone cutting device as shown in FIG. 31B. Then, the bone cutting device may be inserted into the cutting slots 910a and 910b to cut the third bone and the second bone, which prepare the joint between the third bone and the second bone for fusion. After the cuts are made, the third and fourth bone fasteners 976, 978 may be removed to remove the cutting guide 900.

In some examples, the cutting guide assembly (CGA) 600 and the cutting guides 700, 800, 900 may be made of metal (stainless steel or titanium alloy). In other examples, the cutting guide assembly (CGA) 600 and the cutting guides 700, 800, 900 may be made of any other suitable durable material.

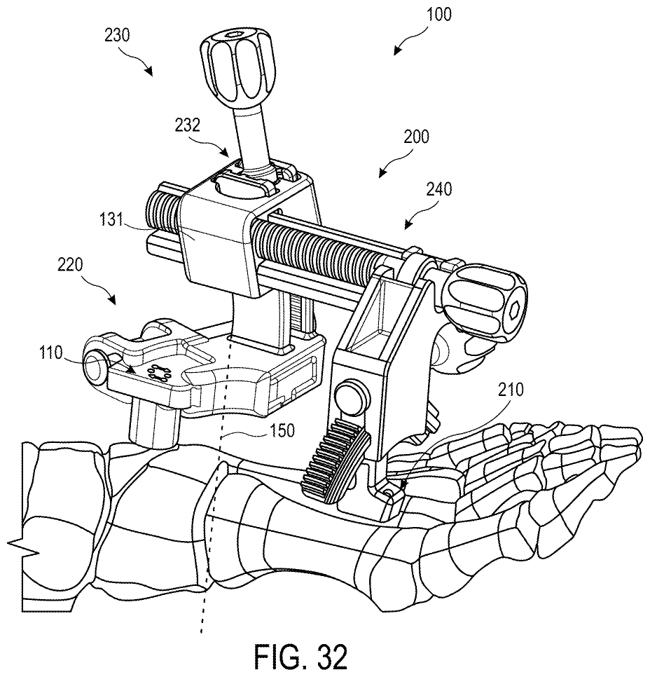

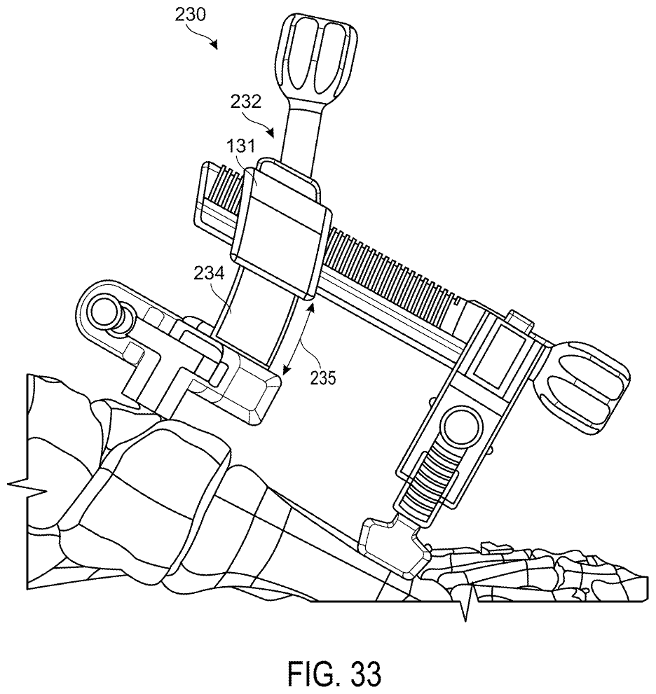

FIGS. 32 and 33 illustrate another example system for controlled realignment of a bone in multiple planes according to another example of the present disclosure. The system may include a main correction assembly 100. In some examples, the main correction assembly 100 may include a fixation body 110 configured to be fixed to a first bone of a patient and a movable body 200 configured to move relative to the fixation body 110. The movable body 200 may include an anchor body 210 configured to be fixed to a second bone of the patient, and a first portion 220 configured to rotate the anchor body 210 relative to the fixation body in a first rotating direction 225 in a first plane. In some examples, the first plane may be a transverse plane.

In some examples, the main correction assembly 100 may define a rotation axis 150 about which the movable body 200 (e.g., anchor body 210 and/or the first portion 220) may be rotated relative to the fixation body 110 in the first plane. In some examples, the main correction assembly 100 may be configured such that the rotation axis 150 is disposed substantially within a joint adjacent the second bone when the fixation body is fixed to the first bone. For example, the rotation axis 150 or at least a portion thereof may be disposed within the joint adjacent the second bone, or a shortest distance between the rotation axis and the joint may be less than about 5 mm, preferably, less about 3 mm, more preferably, less than 1 mm.

In some examples, the movable body 200 may include a second portion 230. The second portion 230 may have a second moving mechanism 232 configured to rotate the anchor body 210 relative to the fixation body 110 in a second direction 235 in a second plane. The second portion 230 may include a second portion body 234. In some examples, the second moving mechanism 232 may further move all components disposed between the second moving mechanism 232 and the anchor body 210 in the second direction 235 in the second plane. In some examples, the second plane may be perpendicular to the first plane. The second plane may be a sagittal (dorsal/plantar) plane or substantially parallel to the sagittal plane (for example, when the main correction assembly 100 is attached to a human body).

In some examples, the second moving mechanism 232 may be a threaded rod, and the second portion body 234 may include internal threads configured to engage with the threaded rod. The rotation of the second moving mechanism 232 may rotate anchor body 210 relative to the fixation body 110 in the second direction 235 in the second plane. In some examples, the rotation of the second moving mechanism 232 may further rotate all components disposed between the second moving mechanism 232 and the anchor body 210 in the second direction 235 in the second plane. For example, a connecting body 131 may be provided between the third portion 240 and the second portion 230. The connecting body 131 may be coupled to the second moving mechanism 232. The connecting body 131 (and ultimately the third portion 240) may move along the second portion body 234 by moving the second moving mechanism 232 along the second portion body 234.

As shown in FIG. 33, in some examples, the second portion body 234 may be curved. Therefore, the second moving mechanism 232 may also rotate along the curve of the second portion body 234. In some examples, the radius of curvature of the second portion body 234 and/or the direction 235 may be in a range of about 5 cm to about 20 cm.

Other configurations/features/characteristics of the main correction assembly 100 of FIGS. 32 and 33 may be similar to and/or same as the ones described above with respect to the main correction assembly 100 illustrated with respect to FIG. 18 and, thus, duplicate description may be omitted.

### Embodiments I

Various aspects of the subject matter described herein are set out in the following numbered embodiments:

Embodiment 1. A system comprises: a main correction assembly comprising: a fixation body configured to be fixed to a first bone of a patient; and a movable body configured to move relative to the fixation body, wherein the movable body comprises: an anchor body configured to be fixed to a second bone of the patient; and a first portion having a first moving mechanism configured to move the anchor body relative to the fixation body in a first direction in a first plane.

Embodiment 2. The system of embodiment 1, wherein the first bone comprises one of an intermediate cuneiform bone, a lateral cuneiform bone, a cuboid bone, and a navicular bone.

Embodiment 3. The system of any one of embodiments 1-2, wherein the second bone comprises a first metatarsal bone.

Embodiment 4. The system of any one of embodiments 1-3, wherein the fixation body comprises a toothed flange and the first moving mechanism comprises a first threaded rod that is rotationally coupled to the toothed flange of the fixation body, wherein a rotation of the first threaded rod rotates the anchor body relative to the fixation body in the first plane.

Embodiment 5. The system of any one of embodiments 1-4, wherein the first plane is a transverse plane.

Embodiment 6. The system of any one of embodiments 1-5, wherein the movable body further comprises a second portion having a second moving mechanism configured to move the anchor body relative to the fixation body in a second direction in a second plane.

Embodiment 7. The system of embodiment 6, wherein the second plane is perpendicular to the first plane.

Embodiment 8. The system of any one of embodiments 6-7, wherein the second plane is a sagittal plane.

Embodiment 9. The system of any one of embodiments 6-8, wherein the second moving mechanism comprises a second portion body and a mover.

Embodiment 10. The system of any one of embodiments 1-9, wherein the movable body further comprises a third portion having a third moving mechanism configured to move the anchor body relative to the fixation body in a third direction.

Embodiment 11. The system of any one of embodiments 10, wherein the third direction is in the first plane.

Embodiment 12. The system of any one of embodiments 10-11, wherein the movement of the anchor body relative to the fixation body in the third direction causes a joint adjacent the second bone to be compressed or distracted.

Embodiment 13. The system of any one of embodiments 10-12, wherein the third moving mechanism comprises a third threaded rod, and the third portion further comprises a third threaded rod support extending in parallel with the third threaded rod.

Embodiment 14. The system of any one of embodiments 1-13, wherein the movable body further comprises a fourth portion having a fourth moving mechanism configured to move the anchor body relative to the fixation body in a fourth direction in a third plane.

Embodiment 15. The system of embodiments 14, wherein the third plane is a frontal plane.

Embodiment 16. The system of any one of embodiments 14-15, wherein the anchor body comprises: a rotating arm rotationally coupled to the fourth moving mechanism; and an anchor base configured to be fixed to the second bone of the patient.

Embodiment 17. The system of embodiment 16, wherein the rotating arm is in a shape of a curve or a partial circle.

Embodiment 18. The system of embodiment 17, wherein the rotating arm is configured such that a center of the curve or the partial circle is located in the second bone when the anchor body is fixed to the second bone.

Embodiment 19. The system of any one of embodiments 16-18, wherein the anchor base extends in the third direction, and the rotating arm extends in a direction perpendicular to the third direction.

Embodiment 20. The system of any one of embodiments 16-19, wherein the anchor base comprises one or more channels configured to receive a bone fastener.

Embodiment 21. The system of any one of embodiments 16-20, wherein the fourth moving mechanism comprises a fourth threaded rod that is rotationally coupled to the rotating arm, wherein a rotation of the fourth threaded rod rotates the anchor body relative to the fixation body in the fourth direction.

Embodiment 22. The system of any one of embodiments 1-21, wherein the fixation body comprises one or more channels configured to receive a bone fastener.

Embodiment 23. The system of embodiment 22, wherein the one or more channels of the fixation body comprise a first channel having a first trajectory, a second channel having a second trajectory, and a third channel having a third trajectory, wherein the first and second trajectories are parallel to each other, and the third trajectory is non-parallel to the first and second trajectories.

Embodiment 24. The system of any one of embodiments 1-23, further comprising an alignment guide configured to be removably coupled to the fixation body of the main correction assembly.

Embodiment 25. The system of embodiment 24, wherein the alignment guide comprises an alignment channel configured to receive a bone fastener, wherein the alignment guide and the main correction assembly are aligned with respect to the first bone and the second bone using the alignment channel and the bone fastener.

Embodiment 26. The system of embodiment 25, wherein the bone fastener is configured to be inserted into a joint adjacent the second bone of the patient.

Embodiment 27. The system of any one of embodiments 25-26, wherein the alignment guide comprises: a first support having a first end portion and a second end portion; a second support having a first end portion and a second end portion, wherein the fixation body is interposed between the first support and the second support when the alignment guide is coupled to the fixation body; a third support having a first end portion connected to the second end portion of the first support and a second end portion connected to the second end portion of the second support; and a securing device configured to secure the alignment guide to the fixation body, wherein the securing device is connected to the first end portion of the first support and the first end portion of the second support.

Embodiment 28. The system of embodiment 27, wherein the alignment channel is disposed in the third support.

Embodiment 29. The system of any one of embodiments 27-28, wherein the alignment guide further comprises a receiver configured to receive a cutting guide configured to guide a bone cutting device, wherein the receiver is disposed at the second end portion of the third support.

Embodiment 30. The system of embodiment 29, wherein the cutting guide comprises one or more cutting slots, wherein the bone cutting device is configured to be inserted into the one or more cutting slots.

Embodiment 31. The system of any one of embodiments 29-30, wherein the cutting guide comprises one or more guiding surfaces, wherein the bone cutting device is configured to be abutted adjacent to the one or more guiding surfaces.

Embodiment 32. The system of any one of embodiments 27-31, wherein the alignment guide further comprises a receiver configured to receive a joint alignment guide, wherein the receiver is disposed at the second end portion of the third support.

Embodiment 33. The system of embodiment 32, wherein the joint alignment guide comprises one or more ridges, wherein the one or more ridges are configured to be aligned with a joint adjacent to the second bone of the patient.

Embodiment 34. A method of using the system of any one of embodiments 1-33, the method comprising: aligning the main correction assembly with respect to the first and second bones of the patient; fixing the fixation body to the first bone of the patient; fixing the anchor body to the second bone of the patient; moving the anchor body, using the first moving mechanism, relative to the fixation body in the first direction in the first plane to correct a position of the second bone in the first direction; and cutting the second bone by a bone cutting device.

Embodiment 35. The method of embodiment 34, further comprising: prior to aligning the main correction assembly, inserting a first bone fastener into a joint adjacent the second bone of the patient, wherein aligning the main correction assembly comprises attaching an alignment guide to the fixation body; and inserting the first bone fastener down through an alignment channel of the alignment guide.

Embodiment 36. The method of embodiment 35, wherein fixing the fixation body to the first bone comprises inserting one or more bone fasteners into the first bone through one or more channels of the fixation body.

Embodiment 37. The method of any one of embodiments 35-36, further comprising removing the first bone fastener by pulling the first bone fastener out of the joint and the alignment channel of the alignment guide.

Embodiment 38. The method of embodiment 37, further comprising removing the alignment guide by detaching the alignment guide from the main correction assembly.

Embodiment 39. The method of any one of embodiments 34-38, wherein fixing the anchor body to the second bone comprises inserting one or more bone fasteners into the second bone through one or more channels of the anchor body.

Embodiment 40. The method of any one of embodiments 34-39, further comprising moving the anchor body, using a second moving mechanism of a second portion of the movable body, relative to the fixation body in a second direction in a second plane to correct the position of the second bone in the second direction.

Embodiment 41. The method of embodiment 40, wherein the second plane is a sagittal plane.

Embodiment 42. The method of any one of embodiments 34-41, further comprising moving the anchor body, using a third moving mechanism of a third portion of the movable body, relative to the fixation body in a third direction.

Embodiment 43. The method of embodiment 42, wherein the movement of the anchor body relative to the fixation body in the third direction causes a joint adjacent the second bone to be compressed or distracted.

Embodiment 44. The method of any one of embodiments 34-43, further comprising moving the anchor body, using a fourth moving mechanism of a fourth portion of the movable body, relative to the fixation body in a fourth direction in a third plane to correct the position of the second bone in the fourth direction.

Embodiment 45. The method of embodiment 44, wherein the third plane is a frontal plane.

Embodiment 46. The method of any one of embodiments 34-45, wherein cutting the second bone comprises inserting the bone cutting device into the second bone along a cutting guide.

Embodiment 47. The method of embodiment 46, wherein the cutting guide comprises one or more cutting slots, and inserting the bone cutting device into the second bone along the cutting guide comprises inserting the bone cutting device into the second bone through one of the cutting slots.

Embodiment 48. The method of any one of embodiments 34-47, further comprising cutting a third bone by inserting the bone cutting device into the third bone along a cutting guide.

Embodiment 49. The method of embodiment 48, wherein the cutting guide comprises one or more cutting slots, and inserting the bone cutting device into the third bone along the cutting guide comprises inserting the bone cutting device into the third bone through one of the cutting slots.

Embodiment 50. The method of any one of embodiments 48-49, wherein the cutting guide comprises one or more guiding surfaces, and inserting the bone cutting device into the third bone along the cutting guide comprises abutting the bone cutting device adjacent to one of the guiding surfaces.

Embodiment 51. The method of any one of embodiments 34-50, wherein aligning the main correction assembly comprises: attaching an alignment guide to the fixation body; inserting a joint alignment guide in a receiver of the alignment guide; and aligning a ridge of the joint alignment guide with a joint adjacent to the second bone.

Embodiment 52. The method of embodiment 51, wherein fixing the fixation body to the first bone comprises inserting one or more bone fastener into the first bone through one or more channels of the fixation body.

Embodiment 53. The method of embodiment 52, further comprising removing the alignment guide and joint alignment guide by detaching the alignment guide from the main correction assembly.

### Embodiments II

Additional various aspects of the subject matter described herein are set out in the following numbered embodiments:

Embodiment 1. A system comprises: a main correction assembly comprising: a fixation body configured to be fixed to a first bone of a patient; a movable body configured to move relative to the fixation body, wherein the movable body comprises: an anchor body configured to be fixed to a second bone of the patient; a first portion configured to rotate the anchor body relative to the fixation body in a first rotating direction in a first plane, wherein the main correction assembly defines a rotation axis about which the anchor body is rotated, by the first portion, relative to the fixation body in the first plane, wherein the main correction assembly is configured such that the rotation axis is disposed substantially within a joint adjacent the second bone when the fixation body is fixed to the first bone.

Embodiment 2. The system of embodiment 1, wherein the first bone comprises one of an intermediate cuneiform bone, a lateral cuneiform bone, a cuboid bone, and a navicular bone.

Embodiment 3. The system of any one of embodiments 1 and 2, wherein the second bone comprises a first metatarsal bone.

Embodiment 4. The system of any one of embodiments 1-3, wherein the joint comprises a first tarsometatarsal joint between a medial cuneiform bone and a first metatarsal bone.

Embodiment 5. The system of any one of embodiments 1-4, wherein the fixation body comprises a curved arm and the first portion is configured to rotate along the curved arm of the fixation body.

Embodiment 6. The system of embodiment 5, wherein the curved arm comprises teeth, and the first portion comprises a first threaded rod that is rotationally coupled to the teeth of the curved arm of the fixation body, wherein a rotation of the first threaded rod along the teeth of the curved arm rotates the anchor body relative to the fixation body in the first plane.

Embodiment 7. The system of any one of embodiments 1-6, wherein the first plane is a transverse plane.

Embodiment 8. The system of any one of embodiments 1-7, wherein the fixation body comprises one or more channels configured to receive a bone fastener so that the fixation body is fixed to the first bone.

Embodiment 9. The system of embodiment 8, wherein the one or more channels of the fixation body comprise a first channel having a first trajectory, a second channel having a second trajectory, and a third channel having a third trajectory, wherein the first and second trajectories are parallel to each other, and the third trajectory is non-parallel to the first and second trajectories.

Embodiment 10. The system of embodiment 9, wherein the one or more channels of the fixation body further comprise a fourth channel having a fourth trajectory, wherein the fourth trajectory is non-parallel to the first, second, and third trajectories.

Embodiment 11. The system of embodiment10, wherein the third and fourth trajectories are symmetrical to each other relative to an axis in parallel with the first and second trajectories.

Embodiment 12. The system of any one of embodiments 10-11, wherein the fixation body comprises one or more visual markers indicating a direction of the third and/or fourth trajectories.

Embodiment 13. The system of any one of embodiments 1-12, wherein the movable body further comprises a second portion configured to move the anchor body relative to the fixation body in a second direction in a second plane.

Embodiment 14. The system of embodiment 13, wherein the second plane is perpendicular to the first plane.

Embodiment 15. The system of any one of embodiments 13-14, wherein the second plane is a sagittal plane.

Embodiment 16. The system of any one of embodiments 13-15, wherein the movable body further comprises a third portion configured to move the anchor body relative to the fixation body in a third direction.

Embodiment 17. The system of embodiment 16, wherein the third direction is parallel with the first plane and/or the second plane.

Embodiment 18. The system of any one of embodiments 16-17, wherein the movement of the anchor body relative to the fixation body in the third direction causes the joint adjacent the second bone to be compressed or distracted.

Embodiment 19. The system of any one of embodiments 16-18, wherein the movable body further comprises a fourth portion configured to move the anchor body relative to the fixation body in a fourth direction in a third plane.

Embodiment 20. The system of embodiment 19, wherein the third plane is a frontal plane.

Embodiment 21. The system of any one of embodiments 1-20, further comprising a cutting guide configured to guide a bone cutting device, wherein the cutting guide comprises one or more cutting slots configured to receive the bone cutting device.

Embodiment 22. The system of embodiment 21, wherein the cutting guide comprises: a first channel configured to receive a first bone fastener to fix the cutting guide to a third bone of the patient; a second channel configured to receive a second bone fastener to fix the cutting guide to the second bone of the patient.

Embodiment 23. The system of embodiment 22, wherein the cutting guide further comprises: a third channel configured to receive a third bone fastener to be inserted into the joint to align the cutting guide with the joint, the second bone, and/or the third bone.

Embodiment 24. The system of embodiment 23, wherein the one or more cutting slots comprise a first cutting slot and a second cutting slot, wherein the third channel is disposed between the first cutting slot and the second cutting slot.

Embodiment 25. The system of embodiment 24, wherein the first cutting slot is provided to guide the bone cutting device to cut the third bone, and the second cutting slot is provided to guide the bone cutting device to cut the second bone.

Embodiment 26. The system of any one of embodiments 22-25, wherein the first channel comprises at least two first channels, and the second channel comprises at least two second channels.

Embodiment 27. The system of any one of embodiment 21-26, wherein at least a portion of the cutting guide and the one or more cutting slots are curved.

Embodiment 28. The system of any one of embodiments 21-27, wherein the cutting guide comprises a top surface and a bottom surface opposite the top surface, wherein the cutting guide further comprises one or more fins protruding from the bottom surface and configured to interact with the joint so that the cutting guide is aligned with the joint.

Embodiment 29. A system comprises: a main correction assembly comprising: a fixation body configured to be fixed to a first bone of a patient; a movable body configured to move relative to the fixation body, wherein the movable body comprises: an anchor body configured to be fixed to a second bone of the patient; a first portion configured to rotate the anchor body relative to the fixation body in a first direction in a first plane; and a cutting guide assembly (CGA) comprising: a first CGA portion configured to be removably coupled to the fixation body of the main correction assembly; a second CGA portion, wherein the first CGA portion is movably coupled to the second CGA portion; a cutting guide configured to guide a bone cutting device, wherein the cutting guide comprises one or more cutting slots configured to receive the bone cutting device, wherein the second CGA portion is movably coupled to the cutting guide.

Embodiment 30. The system of embodiment 29, wherein the first CGA portion comprises an opening, wherein an inner surface of the first CGA portion within the opening is configured to secure to an outer surface of the fixation body.

Embodiment 31. The system of embodiment 30, wherein the first CGA portion is in a ring or hoop shape.

Embodiment 32. The system of any one of embodiments 30-31, wherein the first CGA portion is rotatable relative to the second CGA portion.

Embodiment 33. The system of any one of embodiments 29-32, wherein the second CGA portion comprises an arm with a slot, wherein the first CGA portion is movable relative to the second CGA portion along the slot.

Embodiment 34. The system of any one of embodiments 29-33, wherein the cutting guide assembly further comprises an adjustable fastening mechanism configured to fasten and unfasten the first CGA portion to and from the second CGA portion.

Embodiment 35. The system of embodiment 34, wherein the fastening of the first CGA portion to the second CGA portion by the adjustable fastening mechanism prevents the first CGA portion from moving and rotating relative to the second CGA portion.

Embodiment 36. The system of any one of embodiments 34-35, wherein the adjustable fastening mechanism comprises a cam lock lever.

Embodiment 37. The system of any one of embodiments 34-36, wherein the first CGA portion is configured to move relative to the second CGA portion in a fifth direction, and the second CGA portion is configured to move relative to the cutting guide in a sixth direction, wherein the fifth direction is perpendicular to the sixth direction.

Embodiment 38. The system of embodiment 37, wherein the cutting guide assembly further comprises a connector connecting the second CGA portion with the cutting guide, wherein the cutting guide is configured to move relative to the connector in a seventh direction, wherein the seventh direction is perpendicular to the fifth direction and/or the sixth direction.

Embodiment 39. The system of any one of embodiments 29-38, wherein the cutting guide comprises a distal channel configured to receive a first bone fastener to fix the cutting guide to the second bone.

Embodiment 40. The system of any one of embodiments 29-39, wherein the cutting guide further comprises a central channel configured to receive a second bone fastener to be inserted into a joint adjacent the second bone to align the cutting guide with the joint.

Embodiment 41. The system of embodiment 40, wherein the one or more cutting slots comprise a first cutting slot and a second cutting slot, wherein the central channel is disposed between the first cutting slot and the second cutting slot.

Embodiment 42. The system of embodiment 41, wherein the first cutting slot is provided to guide the bone cutting device to cut a third bone, and the second cutting slot is provided to guide the bone cutting device to cut the second bone.

Embodiment 43. The system of any one of embodiments 29-42, wherein at least a portion of the cutting guide and the one or more cutting slots are curved.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “coupled,” “attachable,” “attached,” “connectable,” “connected,” or any similar terms may include directly or indirectly coupled, directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

Additionally, in describing the components of the present disclosure, there may be terms used like first, second, A, B, (a), and (b). These are solely for the purpose of differentiating one component from the other but not to imply or suggest the substances, order, sequence, or number of the components.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A system comprising:
a main correction assembly comprising:
a fixation body configured to be fixed to a first bone of a patient; and
a movable body configured to move relative to the fixation body, wherein the movable body comprises:
an anchor body configured to be fixed to a second bone of the patient; and
a first portion having a first moving mechanism configured to move the anchor body relative to the fixation body in a first direction in a first plane.

2. The system of claim 1, wherein the first bone comprises one of an intermediate cuneiform bone, a lateral cuneiform bone, a cuboid bone, and a navicular bone, wherein the second bone comprises a first metatarsal bone.

3. The system of claim 1, wherein the main correction assembly defines a rotation axis about which the anchor body is rotated, by the first portion, relative to the fixation body in the first plane, wherein the main correction assembly is configured such that the rotation axis is disposed substantially within a joint adjacent the second bone when the fixation body is fixed to the first bone.

4. The system of claim 1, wherein the first plane is a transverse plane.

5. The system of claim 1, wherein the movable body further comprises a second portion having a second moving mechanism configured to move the anchor body relative to the fixation body in a second direction in a second plane.

6. The system of claim 5, wherein the second plane is perpendicular to the first plane.

7. The system of claim 5, wherein the second plane is a sagittal plane.

8. The system of claim 5, wherein the movable body further comprises a third portion having a third moving mechanism configured to move the anchor body relative to the fixation body in a third direction.

9. The system of claim 8, wherein the movement of the anchor body relative to the fixation body in the third direction causes a joint adjacent the second bone to be compressed or distracted.

10. The system of claim 8, wherein the third moving mechanism comprises a third threaded rod, and the third portion further comprises a third threaded rod support extending in parallel with the third threaded rod.

11. The system of claim 8, wherein the movable body further comprises a fourth portion having a fourth moving mechanism configured to move the anchor body relative to the fixation body in a fourth direction in a third plane.

12. The system of claim 11, wherein the third plane is a frontal plane.

13. The system of claim 11, wherein the anchor body comprises:
a rotating arm rotationally coupled to the fourth moving mechanism; and
an anchor base configured to be fixed to the second bone of the patient.

14. The system of claim 13, wherein the rotating arm is in a shape of a curve or a partial circle.

15. The system of claim 14, wherein the rotating arm is configured such that a center of the curve or the partial circle is located in the second bone when the anchor body is fixed to the second bone.

16. The system of claim 13, wherein the anchor base extends in the third direction, and the rotating arm extends in a direction perpendicular to the third direction.

17. The system of claim 13, wherein the anchor base comprises one or more channels configured to receive a bone fastener.

18. The system of claim 13, wherein the fourth moving mechanism comprises a fourth threaded rod that is rotationally coupled to the rotating arm, wherein a rotation of the fourth threaded rod rotates the anchor body relative to the fixation body in the fourth direction.

19. The system of claim 1, wherein the fixation body comprises one or more channels configured to receive a bone fastener.

20. The system of claim 19, wherein the one or more channels of the fixation body comprise a first channel having a first trajectory, a second channel having a second trajectory, and a third channel having a third trajectory, wherein the first and second trajectories are parallel to each other, and the third trajectory is non-parallel to the first and second trajectories.
